(19) United States
(12) Patent Application Publication
(10) Pub. No.: US 2014/0100666 A1
(43) Pub. Date: Apr. 10, 2014

(54) APPARATUS AND METHOD FOR TREATING OBESITY USING NEUROTOXINS IN CONJUNCTION WITH BARIATRIC PROCEDURES

(71) Applicant: Allergan, Inc., Irvine, CA (US)

(72) Inventor: Gregory F. Brooks, Irvine, CA (US)

(21) Appl. No.: 13/933,765

(22) Filed: Jul. 2, 2013

Related U.S. Application Data
(62) Division of application No. 11/582,775, filed on Oct. 18, 2006, now abandoned.

(51) Int. Cl. A61K 38/48; A61F 5/00

(52) U.S. Cl. CPC A61K 38/4893; A61F 5/003
USPC 623/23.64; 606/191

(57) ABSTRACT

The present invention provides methods for facilitating weight loss in a patient. The methods of the present invention comprise the steps of administering a neurotoxin to a stomach tissue of an obese patient and performing one of several types of bariatric surgeries in the patient, thereby reducing or eliminating unwanted side effects, such as nausea and vomiting.

### CROSS-REFERENCES TO RELATED APPLICATIONS

This application is a Divisional of U.S. application Ser. No. 11/582,775, filed Oct. 18, 2006, incorporated by reference in its entirety.

The present invention relates to methods for facilitating weight loss. In particular, the present invention relates to methods for reducing weight loss by performing a bariatric procedure in conjunction with an administration of a neurotoxin, e.g., a botulinum toxin, at or in the vicinity of the site of the surgical procedure. Numerous procedures may be performed using the method of the present invention, including insertion of an intragastric balloon into the stomach, application of a gastric band around or inside the stomach, or gastric bypass surgery. Those skilled in the art of the invention will recognize that the method of the present invention is not limited to those types of procedures, and that the method of the present invention may be performed in any procedure where the physiology of the stomach is altered or an object is inserted into the stomach. The use of a neurotoxin, e.g., botulinum, lessens the discomfort associated with bariatric procedures by relaxing the stomach muscles and lessening the discomfort associated with the procedure, as well as minimizing and in some cases eliminating unwanted side effects, such as pain and nausea.

Affecting weight loss is one of the key steps in the treatment of obesity. Obesity, especially morbid obesity, is a condition that is associated with a multitude of other hazards to health that include reduced life expectancy and has even been associated with serious sociopsychologic and economic problems.

### Intragastric Balloons

Intragastric balloon systems, such as the BioEnterics® Intragastric Balloon (BIB®) System, are designed as a non-surgical, non-pharmaceutical alternative for the treatment of obesity. Intragastric balloons provide short-term weight loss therapy to reduce health risks related to obesity or risks prior to vital surgery, or as part of a supervised weight loss program. Endoscopically placed and inflated with fluid, such as saline, intragastric balloons (which can be made with durable, elastic, high-quality silicone and other flexible materials) partially fill the stomach to induce the feeling of fullness, and support patients in reducing food intake and adopting new dietary habits. An intragastric balloon may be used in conjunction with a supervised diet and behavior modification program to help maintain weight loss over time after removal of the device, which can also be performed endoscopically. In conjunction with a supervised diet and behavioral modification program, an intragastric balloon can help patients achieve the health and aesthetic benefits associated with weight loss.

Intragastric balloons typically consist of a soft, expandable balloon that a surgeon can orally insert into the patient's stomach without requiring invasive surgery. Once inserted into the stomach, the empty balloon is filled with sterile saline. When full, the balloon is too large to pass into the intestine and will now float freely in the stomach. Use of an intragastric balloon is intended to make compliance with a supervised diet and behavior modification program easier. The balloon partially fills the stomach, and patients report that they have a feeling of satiety or fullness.

The balloon is introduced into the stomach through the mouth without the need for surgery. The physician conducts an initial examination of the stomach using a gastroscopic camera. If no abnormalities are observed, the physician proceeds with placement of the balloon through the mouth, down the esophagus and into the stomach. The balloon is made of a pliable material, such as a silicone elastomer, and is inserted while in its smallest, deflated form. The swallowing process is made easier with the help of anesthetics applied topically to numb the throat area. Muscle relaxing medications may also be used. Once the balloon is inside the stomach, it is immediately filled with sterile saline through a small filling tube, or catheter, attached to the balloon.

Once filled, the doctor removes the filling tube by gently pulling on the external end. The balloon has a self-sealing valve, and at this point the balloon is floating freely in the stomach. Placement times vary, but the total procedure time will usually take 30-60 minutes, after which the patient will be monitored by the physician for a short time and then may return home.

Intragastric balloons can currently be used for approximately six months. Over time, the acidic content of the stomach will weaken the balloon material and cause the balloon to deflate. Should a physician recommend the use of the balloon for longer than six months, it is usually necessary that the balloon be replaced with a new one when the six-month interval has been met. The balloon is normally removed in the same way it was placed, via the esophagus and mouth. Prior to removal, a muscle relaxant may be given with a topical anesthetic to numb the throat. Using a gastroscopic camera, the physician will introduce a catheter through the mouth and into the stomach. The balloon will then be punctured and deflated. Once the balloon is deflated it can be grasped and removed.

One reported problem associated with the current intragastric balloon insertion procedure is that there can be unpleasant effects associated with the insertion of the balloon. For example, the presence of the balloon in the stomach may cause nausea or vomiting for a few days after placement. The physician conventionally prescribes medication to alleviate these potential effects.

### Gastric Bands

Another effective method that has been used to facilitate weight loss includes the deployment of a band around a portion of the stomach creating a stoma opening that is less in diameter than the stomach for restricting food intake into the lower digestive portion of the stomach. The band is commonly called a gastric band. Commercially available gastric bands are sold by Inamed, CA, USA, under the tradename LAP-BAND® System. Alternatively, an intragastric band may be deployed within the stomach to create the desired stoma.

Typically, the band is made of a nonextensible material and is located on the outside of the stomach thereby prohibiting the stoma opening to expand. An important feature of the band deployed around the stomach is that it is adjustable. Adjustment is accomplished by means of a balloon that lines the inside of the band. On the day of surgery, when the band is deployed, the balloon is empty and this provides only a slight restriction to eating. Over the weeks and months following surgery the balloon within the band is gradually filled (outlet is tightened) to provide progressively increasing restriction that is matched or “tuned” to each patient.

The balloon adjustment is accomplished using an access port (which is buried under the skin) to increase or decrease the amount of saline fluid contained in the balloon. This banding procedure itself has been described in articles by Solhaug, “Gastric Banding: A New Method in the Treatment of Morbid Obesity,” Current Surgery, pp. 424-428, November-December 1983; and Check, “Yet Another Variation on Surgery for Obesity,” Journal of the American Medical Association, Vol. 248, No. 16, pp. 1939, 1943, Oct. 22/29, 1982.

There are several key features that make the band an attractive surgical technique for weight loss: laparoscopic deployment, no division or anastomosis of stomach or intestine, removable and adjustable. The first two of the features above probably reduce the risk of surgery, which is especially important when operating on patients who suffer from morbid obesity. The fact that there is no cutting or repositioning of any intestine brings the risk of leak or obstruction to very low levels. The fact that the procedure is almost always done laparoscopically may allow decreased stress on the vital organs (heart, lungs, etc.) and may allow quicker recovery in comparison to open procedures.

“Removable” in the list of key features refers to the fact that the band can be removed from the patient with little residual impact on the stomach. This seems to be true even when the band has eroded into the stomach, or become infected, or slipped out of position. This is possible because the silastic substance from which the band is made creates essentially no tissue reaction, so that the band is not stuck in place over time. This feature also means that the band procedure is “reversible” in a certain sense.

The feature of the band that deserves more attention is that it is adjustable. This is the feature that makes the band (in many published reports) successful in helping patients achieve significant sustained weight loss. After all, if the band were not successful, then the decrease in operative risk would not mean much. As long as the patient and surgeon continue to work together, it is usually possible to adjust the band to the patient's needs at that time.

A major advantage in using the band is that it allows for a slower weight loss. The band aims to create slower and steadier weight loss than the results seen after most other surgical procedures. Most weight loss operations create very rapid weight loss in the first few months, which then slows and stabilizes at 10-18 months after surgery. On the other hand, band patients begin with a relatively loose band that allows ongoing intake of nutrition, and the band is gradually “tightened” according to the patient's weight progress and satiety symptoms. This approach aims to achieve a weight loss of 1-2 pounds per week that continues up to or beyond 30 months after surgery.

The use of a gastric band for facilitating weight loss has great promise due to its simplicity and effectiveness. However, the step of deploying the band around the stomach and/or adjusting (i.e., tightening/loosening) the band may be challenging due to the stiffness of the stomach. Further, after the band is deployed around the upper stomach, the band can slip out of its correct position. If it slips out of position, it is likely to cause obstruction of the stomach, requiring urgent re-operation to reposition the band. In addition, oftentimes patients experience unwanted side effects of nausea and vomiting as a result of the sensation created by the gastric band.

The challenges of deploying the gastric band around the stomach and the risk of the band possibly slipping from its correct position may compromise the full potential use of the gastric band as a technique for affecting weight loss.

### Gastric Bypass Surgery

A gastric bypass consists of a division of the stomach into a small upper pouch and a much larger, lower “remnant” pouch, accompanied by re-arrangement of the small intestines to permit both pouches to remain connected. The manner in which the intestines are reconnected gives rise to several variations of the procedure. The operation leads to a marked reduction in the functional volume of the stomach, accompanied by an altered physiological and psychological response to food. Weight loss using the gastric bypass procedure is typically drastic. There are several different methods for performing the gastric bypass surgery.

A first type of gastric bypass surgery is commonly referred to as a Roux en-Y Proximal procedure. This variant is the most commonly employed gastric bypass technique. In this procedure, the small intestine is divided approximately 18 inches below the lower stomach outlet, and is re-arranged into a Y-configuration, to enable outflow of food from the small upper stomach pouch, via a “Roux limb”. In this procedure, the Y-intersection is formed near the upper (proximal) end of the small intestine. The Roux limb is constructed with a length of approximately 80 to 150 cm (30 to 60 inches), preserving most of the small intestine for absorption of nutrients. The patient experiences very rapid onset of a sense of stomach-fullness.

A second type of gastric bypass surgery is commonly referred to as the Roux en-Y Distal procedure. The normal small intestine is approximately 600 to 1000 cm (20 to 33 feet) in length. As the Y-connection is moved farther down the Gastrointestinal tract, the amount of small intestine capable of fully absorbing nutrients is progressively reduced, in pursuit of greater effectiveness of the operation. The Y-connection is formed much closer to the lower (distal) end of the small intestine, approximately 100 to 150 cm (40 to 60 inches) from the lower end of the intestine, causing reduced absorption of food, primarily of fats and starches, but also of various minerals and fat-soluble vitamins. The unabsorbed fats and starches pass into the large intestine, where bacterial action may act on them to produce irritants and malodorous gases. These nutritional effects are traded for a relatively modest increase in total weight loss.

The gastric bypass reduces the size of the stomach by well over 90%. A normal stomach can stretch, sometimes to over 1000 mL, while the pouch of the gastric bypass may be as small as 15 mL in size. The gastric bypass pouch is usually formed from the part of the stomach that is least susceptible to stretching. That, and its small original size, prevents any significant long-term change in pouch volume.

When the patient ingests just a small amount of food, the first response is stretching of the wall of the small stomach pouch that has been created by the bypass procedure, which stimulates nerves that tell the brain that the stomach is full. The patient feels a sensation of fullness, as if he/she had just eaten a large meal—but with a very small amount of food.

Normally when food is eaten and passed into the stomach, the food passes out of the stomach into the duodenum after only about 30 minutes. When it reaches the lower end of the duodenum, a new hormonal message is generated, telling the brain that enough food has been eaten. The person with a normal gastrointestinal tract experiences this hormone release as a feeling of fullness.

The gastric bypass, when the intestine is re-arranged, moves this portion of the intestine to connect it with the small gastric pouch. The gastric bypass patient, within just a few minutes, and before he or she can eat more than a small amount, begins to feel full.

As with the intragastric balloon and gastric balloon procedures discussed above, it is often difficult for the physician to manipulate the un-relaxed stomach muscles during the bypass procedure. In addition, oftentimes patients experience unwanted side effects of nausea and vomiting as a result of the change of the physiology of the stomach. These challenges may compromise the full potential of the gastric bypass procedure.

### The Stomach

The stomach is an expanded section of the digestive tract between the esophagus and small intestine. The terms used to describe the major regions of the stomach are shown in FIG. 1. The right side of the stomach shown in FIG. 1 is called the greater curvature and that on the left the lesser curvature. The most distal and narrow section of the stomach is termed the pylorus—as food is liquefied in the stomach it passes through the pyloric canal into the small intestine.

The wall of the stomach consists of four coats: serous, muscular, areolar, and mucous, together with vessels and nerves.

The serous coat (tunica serosa) is derived from the peritoneum, and covers the entire surface of the organ, excepting along the greater and lesser curvatures at the points of attachment of the greater and lesser omenta; here the two layers of peritoneum leave a small triangular space, along which the nutrient vessels and nerves pass. On the posterior surface of the stomach, close to the cardiac orifice, there is also a small area uncovered by peritoneum, where the organ is in contact with the under surface of the diaphragm.

The muscular coat (tunica muscularis) (FIGS. 1B and 1C) is situated immediately beneath the serous covering, with which it is closely connected. It consists of three sets of smooth muscle fibers: longitudinal, circular and oblique.

The longitudinal fibers (stratum longitudinale) are the most superficial, and are arranged in two sets. The first set consists of fibers continuous with the longitudinal fibers of the esophagus; they radiate in a stellate manner from the cardiac orifice and are practically all lost before the pyloric portion is reached. The second set commences on the body of the stomach and passes to the right, its fibers becoming more thickly distributed as they approach the pylorus. Some of the more superficial fibers of this set pass on to the duodenum, but the deeper fibers dip inward and interlace with the circular fibers of the pyloric valve.

The circular fibers (stratum circulage) form a uniform layer over the whole extent of the stomach beneath the longitudinal fibers. At the pylorus they are most abundant, and are aggregated into a circular ring, which projects into the lumen, and forms, with the fold of mucous membrane covering its surface, the pyloric valve. They are continuous with the circular fibers of the esophagus, but are sharply marked off from the circular fibers of the duodenum.

The oblique fibers (fibrae obliquae) internal to the circular layer, are limited chiefly to the cardiac end of the stomach, where they are disposed as a thick uniform layer, covering both surfaces, some passing obliquely from left to right, others from right to left, around the cardiac end.

The areolar or submucous coat (tela submucosa) consists of a loose, areolar tissue, connecting the mucous and muscular layers.

The mucous membrane (tunica mucosa) is thick and its surface is smooth, soft, and velvety. In the fresh state it is of a pinkish tinge at the pyloric end, and of a red or reddish-brown color over the rest of its surface. In infancy it is of a brighter hue, the vascular redness being more marked. It is thin at the cardiac extremity, but thicker toward the pylorus. During the contracted state of the organ it is thrown into numerous plaits or ruge, which, for the most part, have a longitudinal direction, and are most marked toward the pyloric end of the stomach, and along the greater curvature. These folds are entirely obliterated when the organ becomes distended.

### Botulinum Toxin

The genus Clostridium has more than one hundred and twenty seven species, grouped according to their morphology and functions. The anaerobic, gram positive bacterium Clostridium botulinum produces a potent polypeptide Clostridial toxin, botulinum toxin, which causes a neuroparalytic illness in humans and animals referred to as botulism. The spores of Clostridium botulinum are found in soil and can grow in improperly sterilized and sealed food containers of home based canneries, which are the cause of many of the cases of botulism. The effects of botulism typically appear 18 to 36 hours after eating the foodstuffs infected with a Clostridium botulinum culture or spores. The botulinum toxin can apparently pass unattenuated through the lining of the gut and attack peripheral motor neurons. Symptoms of botulinum toxin intoxication can progress from difficulty walking, swallowing, and speaking to paralysis of the respiratory muscles and death.

Botulinum toxin type A is the most lethal natural biological agent known to man. About 50 picograms of a commercially available botulinum toxin type A (purified Clostridial toxin complex)¹ is a LD₅₀ in mice (i.e. 1 unit). One unit of BOTOX® contains about 50 picograms (about 56 attomoles) of botulinum toxin type A complex. Interestingly, on a molar basis, botulinum toxin type A is about 1.8 billion times more lethal than diphtheria, about 600 million times more lethal than sodium cyanide, about 30 million times more lethal than cobra toxin and about 12 million times more lethal than cholera. Singh, Critical Aspects of Bacterial Protein Toxins, pages 63-84 (chapter 4) of Natural Toxins II, edited by B. R. Singh et al., Plenum Press, New York (1996) (where the stated LD₅₀ of botulinum toxin type A of 0.3 ng equals 1 U is corrected for the fact that about 0.05 ng of BOTOX® equals 1 unit). One unit (U) of botulinum toxin is defined as the LD₅₀ upon intraperitoneal injection into female Swiss Webster mice weighing 18 to 20 grams each. ¹Available from Allergan, Inc., of Irvine, Calif. under the tradename BOTOX® in 100 unit vials

Seven immunologically distinct botulinum Clostridial toxins have been characterized, these being respectively botulinum Clostridial toxin serotypes A, B, C₁, D, E, F and G each of which is distinguished by neutralization with type-specific antibodies. The different serotypes of botulinum toxin vary in the animal species that they affect and in the severity and duration of the paralysis they evoke. For example, it has been determined that botulinum toxin type A is 500 times more potent, as measured by the rate of paralysis produced in the rat, than is botulinum toxin type B. Additionally, botulinum toxin type B has been determined to be non-toxic in primates at a dose of 480 U/kg which is about 12 times the primate LD₅₀ for botulinum toxin type A. Moyer E et al., Botulinum Toxin Type B: Experimental and Clinical Experience, chapter 6, pages 71-85 of “Therapy With Botulinum Toxin”, edited by Jankovic, J. et al. (1994), Marcel Dekker, Inc. Botulinum toxin apparently binds with high affinity to cholinergic motor neurons, is translocated into the neuron and blocks the release of acetylcholine.

Regardless of serotype, the molecular mechanism of toxin intoxication appears to be similar and to involve at least three steps or stages. In the first step of the process, the toxin binds to the presynaptic membrane of the target neuron through a specific interaction between the heavy chain, H chain, and a cell surface receptor; the receptor is thought to be different for each type of botulinum toxin and for tetanus toxin. The carboxyl end segment of the H chain, H_{C}, appears to be important for targeting of the toxin to the cell surface.

In the second step, the toxin crosses the plasma membrane of the poisoned cell. The toxin is first engulfed by the cell through receptor-mediated endocytosis, and an endosome containing the toxin is formed. The toxin then escapes the endosome into the cytoplasm of the cell. This step is thought to be mediated by the amino end segment of the H chain, H_{N}, which triggers a conformational change of the toxin in response to a pH of about 5.5 or lower. Endosomes are known to possess a proton pump which decreases intra-endosomal pH. The conformational shift exposes hydrophobic residues in the toxin, which permits the toxin to embed itself in the endosomal membrane. The toxin (or at a minimum the light chain) then translocates through the endosomal membrane into the cytoplasm.

The last step of the mechanism of botulinum toxin activity appears to involve reduction of the disulfide bond joining the heavy chain, H chain, and the light chain, L chain. The entire toxic activity of botulinum and tetanus toxins is contained in the L chain of the holotoxin; the L chain is a zinc (Zn++) endopeptidase which selectively cleaves proteins essential for recognition and docking of neurotransmitter-containing vesicles with the cytoplasmic surface of the plasma membrane, and fusion of the vesicles with the plasma membrane. Tetanus Clostridial toxin, botulinum toxin types B, D, F, and G cause degradation of synaptobrevin (also called vesicle-associated membrane protein (VAMP)), a synaptosomal membrane protein. Most of the VAMP present at the cytoplasmic surface of the synaptic vesicle is removed as a result of any one of these cleavage events. Botulinum toxin serotype A and E cleave SNAP-25. Botulinum toxin serotype C₁ was originally thought to cleave syntaxin, but was found to cleave syntaxin and SNAP-25. Each of the botulinum toxins specifically cleaves a different bond, except botulinum toxin type B (and tetanus toxin) which cleave the same bond.

Although all the botulinum toxins serotypes apparently inhibit release of the neurotransmitter acetylcholine at the neuromuscular junction, they do so by affecting different neurosecretory proteins and/or cleaving these proteins at different sites. For example, botulinum types A and E both cleave the 25 kiloDalton (kD) synaptosomal associated protein (SNAP-25), but they target different amino acid sequences within this protein. Botulinum toxin types B, D, F and G act on vesicle-associated protein (VAMP, also called synaptobrevin), with each serotype cleaving the protein at a different site. Finally, botulinum toxin type C₁ has been shown to cleave both syntaxin and SNAP-25. These differences in mechanism of action may affect the relative potency and/or duration of action of the various botulinum toxin serotypes. Apparently, a substrate for a botulinum toxin can be found in a variety of different cell types. See e.g. Gonelle-Gispert, C., et al., SNAP-25a and −25b isoforms are both expressed in insulin-secreting cells and can function in insulin secretion, Biochem J. 1; 339 (pt 1):159-65: 1999, and Boyd R. S. et al., The effect of botulinum Clostridial toxin-B on insulin release from a ∃-cell line, and Boyd R. S. et al., The insulin secreting ∃-cell line, HIT-15, contains SNAP-25 which is a target for botulinum Clostridial toxin-A, both published at Mov Disord, 10(3):376: 1995 (pancreatic islet B cells contains at least SNAP-25 and synaptobrevin).

The molecular weight of the botulinum toxin protein molecule, for all seven of the known botulinum toxin serotypes, is about 150 kD. Interestingly, the botulinum toxins are released by Clostridial bacterium as complexes comprising the 150 kD botulinum toxin protein molecule along with associated non-toxin proteins. Thus, the botulinum toxin type A complex can be produced by Clostridial bacterium as 900 kD, 500 kD and 300 kD forms. Botulinum toxin types B and C₁ are apparently produced as only a 700 kD or 500 kD complex. Botulinum toxin type D is produced as both 300 kD and 500 kD complexes. Finally, botulinum toxin types E and F are produced as only approximately 300 kD complexes. The complexes (i.e. molecular weight greater than about 150 kD) are believed to contain a non-toxin hemaglutinin protein and a non-toxin and non-toxic nonhemaglutinin protein. These two non-toxin proteins (which along with the botulinum toxin molecule comprise the relevant Clostridial toxin complex) may act to provide stability against denaturation to the botulinum toxin molecule and protection against digestive acids when toxin is ingested. Additionally, it is possible that the larger (greater than about 150 kD molecular weight) botulinum toxin complexes may result in a slower rate of diffusion of the botulinum toxin away from a site of intramuscular injection of a botulinum toxin complex.

All the botulinum toxin serotypes are made by Clostridium botulinum bacteria as inactive single chain proteins which must be cleaved or nicked by proteases to become neuroactive. The bacterial strains that make botulinum toxin serotypes A and G possess endogenous proteases and serotypes A and G can therefore be recovered from bacterial cultures in predominantly their active form. In contrast, botulinum toxin serotypes C₁, D, and E are synthesized by nonproteolytic strains and are therefore typically unactivated when recovered from culture. Serotypes B and F are produced by both proteolytic and nonproteolytic strains and therefore can be recovered in either the active or inactive form. However, even the proteolytic strains that produce, for example, the botulinum toxin type B serotype only cleave a portion of the toxin produced. The exact proportion of nicked to unnicked molecules depends on the length of incubation and the temperature of the culture. Therefore, a certain percentage of any preparation of, for example, the botulinum toxin type B toxin is likely to be inactive, possibly accounting for a lower potency of botulinum toxin type B as compared to botulinum toxin type A. The presence of inactive botulinum toxin molecules in a clinical preparation will contribute to the overall protein load of the preparation, which has been linked to increased antigenicity, without contributing to its clinical efficacy.

Botulinum toxins and toxin complexes can be obtained from, for example, List Biological Laboratories, Inc., Campbell, Calif.; the Centre for Applied Microbiology and Research, Porton Down, U.K.; Wako (Osaka, Japan), as well as from Sigma Chemicals of St Louis, Mo. Commercially available botulinum toxin containing pharmaceutical compositions include BOTOX® (Botulinum toxin type A Clostridial toxin complex with human serum albumin and sodium chloride) available from Allergan, Inc., of Irvine, Calif. in 100 unit vials as a lyophilized powder to be reconstituted with 0.9% sodium chloride before use), Dysport® (Clostridium botulinum type A toxin haemagglutinin complex with human serum albumin and lactose in the formulation), available from Ipsen Limited, Berkshire, U.K. as a powder to be reconstituted with 0.9% sodium chloride before use), and MyoBloc™ (an injectable solution comprising botulinum toxin type B, human serum albumin, sodium succinate, and sodium chloride at about pH 5.6, available from Elan Corporation, Dublin, Ireland).

The success of botulinum toxin type A to treat a variety of clinical conditions has led to interest in other botulinum toxin serotypes. Additionally, pure botulinum toxin has been used to treat humans. see e.g. Kohl A., et al., Comparison of the effect of botulinum toxin A (BOTOX®) with the highly-purified Clostridial toxin (NT 201) in the extensor digitorum brevis muscle test, Mov Disord 2000; 15(Suppl 3):165. Hence, a pharmaceutical composition can be prepared using a pure botulinum toxin.

The type A botulinum toxin is known to be soluble in dilute aqueous solutions at pH 4-6.8. At pH above about 7 the stabilizing nontoxic proteins dissociate from the Clostridial toxin, resulting in a gradual loss of toxicity, particularly as the pH and temperature rise. Schantz E. J., et al Preparation and characterization of botulinum toxin type A for human treatment (in particular pages 44-45), being chapter 3 of Jankovic, J., et al, Therapy with Botulinum Toxin, Marcel Dekker, Inc (1994).

The botulinum toxin molecule (about 150 kDa), as well as the botulinum toxin complexes (about 300-900 kDa), such as the toxin type A complex are also extremely susceptible to denaturation due to surface denaturation, heat, and alkaline conditions. Inactivated toxin forms toxoid proteins which may be immunogenic. The resulting antibodies can render a patient refractory to toxin injection.

In vitro studies have indicated that botulinum toxin inhibits potassium cation induced release of both acetylcholine and norepinephrine from primary cell cultures of brainstem tissue. Additionally, it has been reported that botulinum toxin inhibits the evoked release of both glycine and glutamate in primary cultures of spinal cord neurons and that in brain synaptosome preparations botulinum toxin inhibits the release of each of the neurotransmitters acetylcholine, dopamine, norepinephrine (Habermann E., et al., Tetanus Toxin and Botulinum A and C Clostridial toxins Inhibit Noradrenaline Release From Cultured Mouse Brain, J Neurochem 51(2); 522-527: 1988) CGRP, substance P and glutamate (Sanchez-Prieto, J., et al., Botulinum Toxin A Blocks Glutamate Exocytosis From Guinea Pig Cerebral Cortical Synaptosomes, Eur J. Biochem 165; 675-681: 1987. Thus, when adequate concentrations are used, stimulus-evoked release of most neurotransmitters is blocked by botulinum toxin. See e.g. Pearce, L. B., Pharmacologic Characterization of Botulinum Toxin For Basic Science and Medicine, Toxicon 35(9); 1373-1412 at 1393; Bigalke H., et al., Botulinum A Clostridial toxin Inhibits Non-Cholinergic Synaptic Transmission in Mouse Spinal Cord Neurons in Culture, Brain Research 360; 318-324: 1985; Habermann E., Inhibition by Tetanus and Botulinum A Toxin of the release of [³H] Noradrenaline and [³H] GABA From Rat Brain Homogenate, Experientia 44; 224-226: 1988, Bigalke H., et al., Tetanus Toxin and Botulinum A Toxin Inhibit Release and Uptake of Various Transmitters, as Studied with Particulate Preparations From Rat Brain and Spinal Cord, Naunyn-Schmiedeberg's Arch Pharmacol 316; 244-251: 1981, and; Jankovic J. et al., Therapy With Botulinum Toxin, Marcel Dekker, Inc., (1994), page 5.

Botulinum toxin type A can be obtained by establishing and growing cultures of Clostridium botulinum in a fermenter and then harvesting and purifying the fermented mixture in accordance with known procedures. All the botulinum toxin serotypes are initially synthesized as inactive single chain proteins which must be cleaved or nicked by proteases to become neuroactive. The bacterial strains that make botulinum toxin serotypes A and G possess endogenous proteases and serotypes A and G can therefore be recovered from bacterial cultures in predominantly their active form. In contrast, botulinum toxin serotypes C₁, D and E are synthesized by nonproteolytic strains and are therefore typically unactivated when recovered from culture. Serotypes B and F are produced by both proteolytic and nonproteolytic strains and therefore can be recovered in either the active or inactive form. However, even the proteolytic strains that produce, for example, the botulinum toxin type B serotype only cleave a portion of the toxin produced. The exact proportion of nicked to unnicked molecules depends on the length of incubation and the temperature of the culture. Therefore, a certain percentage of any preparation of, for example, the botulinum toxin type B toxin is likely to be inactive, possibly accounting for the known significantly lower potency of botulinum toxin type B as compared to botulinum toxin type A. The presence of inactive botulinum toxin molecules in a clinical preparation will contribute to the overall protein load of the preparation, which has been linked to increased antigenicity, without contributing to its clinical efficacy. Additionally, it is known that botulinum toxin type B has, upon intramuscular injection, a shorter duration of activity and is also less potent than botulinum toxin type A at the same dose level.

High quality crystalline botulinum toxin type A can be produced from the Hall A strain of Clostridium botulinum with characteristics of ≧3×10⁷ U/mg, an A₂₆₀/A₂₇₈ of less than 0.60 and a distinct pattern of banding on gel electrophoresis. The known Schantz process can be used to obtain crystalline botulinum toxin type A, as set forth in Schantz, E. J., et al, Properties and use of Botulinum toxin and Other Microbial Clostridial toxins in Medicine, Microbiol Rev. 56; 80-99: 1992. Generally, the botulinum toxin type A complex can be isolated and purified from an anaerobic fermentation by cultivating Clostridium botulinum type A in a suitable medium. The known process can also be used, upon separation out of the non-toxin proteins, to obtain pure botulinum toxins, such as for example: purified botulinum toxin type A with an approximately 150 kD molecular weight with a specific potency of 1−2×10⁸ LD₅₀ U/mg or greater; purified botulinum toxin type B with an approximately 156 kD molecular weight with a specific potency of 1−2×10⁸ LD₅₀ U/mg or greater, and; purified botulinum toxin type F with an approximately 155 kD molecular weight with a specific potency of 1−2×10⁷ LD₅₀ U/mg or greater.

Either the pure botulinum toxin (i.e. the 150 kilodalton botulinum toxin molecule) or the toxin complex can be used to prepare a pharmaceutical composition. Both molecule and complex are susceptible to denaturation due to surface denaturation, heat, and alkaline conditions. Inactivated toxin forms toxoid proteins which may be immunogenic. The resulting antibodies can render a patient refractory to toxin injection.

As with enzymes generally, the biological activities of the botulinum toxins (which are intracellular peptidases) are dependant, at least in part, upon their three dimensional conformation. Thus, botulinum toxin type A is detoxified by heat, various chemicals, surface stretching and surface drying. Additionally, it is known that dilution of the toxin complex obtained by the known culturing, fermentation and purification to the much, much lower toxin concentrations used for pharmaceutical composition formulation results in rapid detoxification of the toxin unless a suitable stabilizing agent is present. Dilution of the toxin from milligram quantities to a solution containing nanograms per milliliter presents significant difficulties because of the rapid loss of specific toxicity upon such great dilution. Since the toxin may be used months or years after the toxin-containing pharmaceutical composition is formulated, the toxin can be stabilized with a stabilizing agent such as albumin and gelatin.

A commercially available botulinum toxin-containing pharmaceutical composition is sold under the trademark BOTOX® (available from Allergan, Inc., of Irvine, Calif.). BOTOX® consists of a purified botulinum toxin type A complex, albumin and sodium chloride packaged in sterile, vacuum-dried form. The botulinum toxin type A is made from a culture of the Hall strain of Clostridium botulinum grown in a medium containing N-Z amine and yeast extract. The botulinum toxin type A complex is purified from the culture solution by a series of acid precipitations to a crystalline complex consisting of the active high molecular weight toxin protein and an associated hemagglutinin protein. The crystalline complex is re-dissolved in a solution containing saline and albumin and sterile filtered (0.2 microns) prior to vacuum-drying. The vacuum-dried product is stored in a freezer at or below −5° C. BOTOX® can be reconstituted with sterile, non-preserved saline prior to intramuscular injection. Each vial of BOTOX® contains about 100 units (U) of Clostridium botulinum toxin type A purified Clostridial toxin complex, 0.5 milligrams of human serum albumin and 0.9 milligrams of sodium chloride in a sterile, vacuum-dried form without a preservative.

To reconstitute vacuum-dried BOTOX®, sterile normal saline without a preservative; (0.9% Sodium Chloride Injection) is used by drawing up the proper amount of diluent in the appropriate size syringe. Since BOTOX® may be denatured by bubbling or similar violent agitation, the diluent is gently injected into the vial. For sterility reasons BOTOX® is preferably administered within four hours after the vial is removed from the freezer and reconstituted. During these four hours, reconstituted BOTOX® can be stored in a refrigerator at about 2° C. to about 8° C. Reconstituted, refrigerated BOTOX® has been reported to retain its potency for at least about two weeks. Neurology, 48:249-53: 1997.

Botulinum toxins have been used in clinical settings for the treatment of neuromuscular disorders characterized by hyperactive skeletal muscles. Botulinum toxin type A (BOTOX®) was approved by the U.S. Food and Drug Administration in 1989 for the treatment of essential blepharospasm, strabismus and hemifacial spasm in patients over the age of twelve. In 2000 the FDA approved commercial preparations of type A (BOTOX®) and type B botulinum toxin (MyoBloc™) serotypes for the treatment of cervical dystonia, and in 2002 the FDA approved a type A botulinum toxin (BOTOX®) for the cosmetic treatment of certain hyperkinetic (glabellar) facial wrinkles Clinical effects of peripheral intramuscular botulinum toxin type A are usually seen within one week of injection and sometimes within a few hours. The typical duration of symptomatic relief (i.e. flaccid muscle paralysis) from a single intramuscular injection of botulinum toxin type A can be about three months, although in some cases the effects of a botulinum toxin induced denervation of a gland, such as a salivary gland, have been reported to last for several years. For example, it is known that botulinum toxin type A can have an efficacy for up to 12 months (Naumann M., et al., Botulinum toxin type A in the treatment of focal, axillary and palmar hyperhidrosis and other hyperhidrotic conditions, European J. Neurology 6 (Supp 4): S111-S115: 1999), and in some circumstances for as long as 27 months. Ragona, R. M., et al., Management of parotid sialocele with botulinum toxin, The Laryngoscope 109:1344-1346: 1999. However, the usual duration of an intramuscular injection of BOTOX® is typically about 3 to 4 months.

It has been reported that a botulinum toxin type A has been used in diverse clinical settings, including for example as follows:

(1) about 75-125 units of BOTOX® per intramuscular injection (multiple muscles) to treat cervical dystonia;

(2) 5-10 units of BOTOX® per intramuscular injection to treat glabellar lines (brow furrows) (5 units injected intramuscularly into the procerus muscle and 10 units injected intramuscularly into each corrugator supercilii muscle);

(3) about 30-80 units of BOTOX® to treat constipation by intrasphincter injection of the puborectalis muscle;

(4) about 1-5 units per muscle of intramuscularly injected BOTOX® to treat blepharospasm by injecting the lateral pre-tarsal orbicularis oculi muscle of the upper lid and the lateral pre-tarsal orbicularis oculi of the lower lid.

(5) to treat strabismus, extraocular muscles have been injected intramuscularly with between about 1-5 units of BOTOX®, the amount injected varying based upon both the size of the muscle to be injected and the extent of muscle paralysis desired (i.e. amount of diopter correction desired).

(6) to treat upper limb spasticity following stroke by intramuscular injections of BOTOX® into five different upper limb flexor muscles, as follows:

(a) flexor digitorum profundus: 7.5 U to 30 U

(b) flexor digitorum sublimus: 7.5 U to 30 U

(c) flexor carpi ulnaris: 10 U to 40 U

(d) flexor carpi radialis: 15 U to 60 U (e) biceps brachii: 50 U to 200 U. Each of the five indicated muscles has been injected at the same treatment session, so that the patient receives from 90 U to 360 U of upper limb flexor muscle BOTOX® by intramuscular injection at each treatment session.

(7) to treat migraine, pericranial injected (injected symmetrically into glabellar, frontalis and temporalis muscles) injection of 25 U of BOTOX® has showed significant benefit as a prophylactic treatment of migraine compared to vehicle as measured by decreased measures of migraine frequency, maximal severity, associated vomiting and acute medication use over the three month period following the 25 U injection.

Additionally, intramuscular botulinum toxin has been used in the treatment of tremor in patients with Parkinson's disease, although it has been reported that results have not been impressive. Marjama-Lyons, J., et al., Tremor-Predominant Parkinson's Disease, Drugs & Aging 16(4); 273-278: 2000.

Treatment of certain gastrointestinal and smooth muscle disorders with a botulinum toxin are known. See e.g. U.S. Pat. Nos. 5,427,291 and 5,674,205 (Pasricha). Additionally, transurethral injection of a botulinum toxin into a bladder sphincter to treat a urination disorder is known (see e.g. Dykstra, D. D., et al, Treatment of detrusor-sphincter dyssynergia with botulinum A toxin: A double-blind study, Arch Phys Med Rehabil 1990 January; 71:24-6), as is injection of a botulinum toxin into the prostate to treat prostatic hyperplasia. See e.g. U.S. Pat. No. 6,365,164 (Schmidt).

U.S. Pat. No. 5,766,605 (Sanders) proposes the treatment of various autonomic disorders, such as excessive stomach acid secretion, hypersalivation and rhinittis, with a botulinum toxin. Additionally, it is known that nasal hypersecretion is predominantly caused by over activity of nasal glands, which are mainly under cholinergic control and it has been demonstrated that application of botulinum toxin type A to mammalian nasal mucosal tissue of the maxillary sinus turbinates can induce a temporary apoptosis in the nasal glands. Rohrbach S., et al., Botulinum toxin type A induces apoptosis in nasal glands of guinea pigs, Ann Otol Rhinol Laryngol 2001 November; 110(11):1045-50. Furthermore, local application of botulinum toxin A to a human female patient with intrinsic rhinitis resulted in a clear decrease of the nasal hypersecretion within five days. Rohrbach S., et al., Minimally invasive application of botulinum toxin type A in nasal hypersecretion, J Oto-Rhino-Laryngol 2001 November-December; 63(6):382-4.

Various afflictions, such as hyperhydrosis and headache, treatable with a botulinum toxin are discussed in WO 95/17904 (PCT/US94/14717) (Aoki). EP 0 605 501 B1 (Graham) discusses treatment of cerebral palsy with a botulinum toxin, and U.S. Pat. No. 6,063,768 (First) discusses treatment of neurogenic inflammation with a botulinum toxin.

In addition to having pharmacologic actions at the peripheral location, botulinum toxins can also have inhibitory effects in the central nervous system. Work by Weigand et al, (¹²⁵I-labelled botulinum A Clostridial toxin:pharmacokinetics in cats after intramuscular injection, Nauny-Schmiedeberg's Arch. Pharmacol. 1976; 292, 161-165), and Habermann, (¹²⁵I-labelled Clostridial toxin from clostridium botulinum A: preparation, binding to synaptosomes and ascent to the spinal cord, Nauny-Schmiedeberg's Arch. Pharmacol. 1974; 281, 47-56) showed that botulinum toxin is able to ascend to the spinal area by retrograde transport. As such, a botulinum toxin injected at a peripheral location, for example intramuscularly, may be retrograde transported to the spinal cord.

In vitro studies have indicated that botulinum toxin inhibits potassium cation induced release of both acetylcholine and norepinephrine from primary cell cultures of brainstem tissue. Additionally, it has been reported that botulinum toxin inhibits the evoked release of both glycine and glutamate in primary cultures of spinal cord neurons and that in brain synaptosome preparations botulinum toxin inhibits the release of each of the neurotransmitters acetylcholine, dopamine, norepinephrine, CGRP and glutamate.

U.S. Pat. No. 5,989,545 discloses that a modified Clostridial toxin or fragment thereof, preferably a botulinum toxin, chemically conjugated or recombinantly fused to a particular targeting moiety can be used to treat pain by administration of the agent to the spinal cord.

A botulinum toxin has also been proposed for the treatment of hyperhydrosis (excessive sweating, U.S. Pat. No. 5,766,605), headache, (U.S. Pat. No. 6,458,365), migraine headache (U.S. Pat. No. 5,714,468), post-operative pain and visceral pain (U.S. Pat. No. 6,464,986), pain by intraspinal administration (U.S. Pat. No. 6,113,915), Parkinson's disease by intracranial administration (U.S. Pat. No. 6,306,403), hair growth and hair retention (U.S. Pat. No. 6,299,893), psoriasis and dermatitis (U.S. Pat. No. 5,670,484), injured muscles (U.S. Pat. No. 6,423,319), various cancers (U.S. Pat. No. 6,139,845), pancreatic disorders (U.S. Pat. No. 6,143,306), smooth muscle disorders (U.S. Pat. No. 5,437,291, including injection of a botulinum toxin into the upper and lower esophageal, pyloric and anal sphincters)), prostate disorders (U.S. Pat. No. 6,365,164), inflammation, arthritis and gout (U.S. Pat. No. 6,063,768), juvenile cerebral palsy (U.S. Pat. No. 6,395,277), inner ear disorders (U.S. Pat. No. 6,265,379), thyroid disorders (U.S. Pat. No. 6,358,513), parathyroid disorders (U.S. Pat. No. 6,328,977). Additionally, controlled release toxin implants are known (U.S. Pat. Nos. 6,306,423 and 6,312,708). These patents are incorporated in their entirety herein by reference.

It has been reported that that intravenous injection of a botulinum toxin causes a decline of pentagastrin stimulated acid and pepsin secretion in rats. Kondo T., et al., Modification of the action of pentagastrin on acid secretion by botulinum toxin, Experientia 1977; 33:750-1. Additionally it has been speculated that a botulinum toxin can be used to reduce a gastrointestinal secretion, such as a gastric secretion. See pages 16-17 of WO 95/17904. Furthermore, a botulinum toxin has been proposed for the treatment of disorders of gastrointestinal muscle in the enteric nervous system disorder (U.S. Pat. No. 5,437,291) and well as to treat various autonomic disorders (U.S. Pat. No. 5,766,605). Botulinum toxin has been injected into the fundus of the stomach of dogs. Wang Z., et al., Effects of botulinum toxin on gastric myoelectrical and vagal activities in dogs, Gastroenterology 2001 April; 120(5 Suppl 1):A-718. Additionally, intramuscular injection of a botulinum toxin into the gastric antrum has been proposed as a treatment for obesity. See e.g. Gui D., et al., Effects of botulinum toxin on gastric emptying and digestive secretions. A possible tool for correction of obesity?, Naunyn Schmiedebergs Arch Pharmacol 2002 June; 365(Suppl 2):R22; Albanese A., et al., The use of botulinum toxin on smooth muscles, Eur J Neurol 1995 November; 2(Supp 3):29-33, and; Gui D., et al., Botulinum toxin injected in the gastric wall reduces body weight and food intake in rats, Aliment Pharmacol Ther 2000 June; 14(6):829-834. Furthermore, botulinum toxin type A has been proposed as a therapeutic application for the control of secretion in the stomach. Rossi S., et al., Immunohistochemical localization of SNAP-25 protein in the stomach of rat, Naunyn Schmiedebergs Arch Pharmacol 2002; 365(Suppl 2):R37.

Significantly, it has been reported that injection of a botulinum toxin into the lower esophageal sphincter for the treatment of achalasia results in the formation of ulcers in the esophagus. Eaker, E. Y., et al., Untoward effects of esophageal botulinum toxin injection in the treatment of achalasia, Dig Dis Sci 1997 April; 42(4):724-7. It is known to inject a botulinum toxin into a spastic pyloric sphincter of a patient with a prepyloric ulcer in order to permit the pyloric muscle to open. Wiesel P. H. et al., Botulinum toxin for refractory postoperative pyloric spasm, Endoscopy 1997; 29(2): 132.

It is known to inject a botulinum toxin into the stomach wall of a patient to treat obesity by reducing stomach muscle contractions (see e.g. Rolnik J., et al., Antral Injections of botulinum toxin for the treatment of obesity, Ann Intern Med 2003 Feb., 18; 138(4):359-360; 2003, Miller L., WO 02/13854 A1, Obesity controlling method, published Feb. 21, 2002; Gui, D. et al., Botulinum toxin injected in the gastric wall reduces body weight and food intake in rats, Aliment Pharmacol Ther 2000 June; 14(6):829-834; Gui D. et al., Effects of botulinum toxin on gastric emptying and digestive secretions. A possible tool for correction of obesity?, Naunyn Schmiedebergs Arch Pharmacol 2002 June; 365(Suppl 2): R22; Albanese A., et al., The use of botulinum toxin on smooth muscles, Eur J Neurol 1995 November; 2 (Supp 3): 29-33; Albanese A. et al., Review article: the use of botulinum toxin in the alimentary tract, Ailment Pharmacol Ther 1995; 9: 599-604.

Tetanus toxin, as wells as derivatives (i.e. with a non-native targeting moiety), fragments, hybrids and chimeras thereof can also have therapeutic utility. The tetanus toxin bears many similarities to the botulinum toxins. Thus, both the tetanus toxin and the botulinum toxins are polypeptides made by closely related species of Clostridium (Clostridium tetani and Clostridium botulinum, respectively). Additionally, both the tetanus toxin and the botulinum toxins are dichain proteins composed of a light chain (molecular weight about 50 kD) covalently bound by a single disulfide bond to a heavy chain (molecular weight about 100 kD). Hence, the molecular weight of tetanus toxin and of each of the seven botulinum toxins (non-complexed) is about 150 kD. Furthermore, for both the tetanus toxin and the botulinum toxins, the light chain bears the domain which exhibits intracellular biological (protease) activity, while the heavy chain comprises the receptor binding (immunogenic) and cell membrane translocational domains.

Further, both the tetanus toxin and the botulinum toxins exhibit a high, specific affinity for gangliocide receptors on the surface of presynaptic cholinergic neurons. Receptor mediated endocytosis of tetanus toxin by peripheral cholinergic neurons results in retrograde axonal transport, blocking of the release of inhibitory neurotransmitters from central synapses and a spastic paralysis. Contrarily, receptor mediated endocytosis of botulinum toxin by peripheral cholinergic neurons results in little if any retrograde transport, inhibition of acetylcholine exocytosis from the intoxicated peripheral motor neurons and a flaccid paralysis.

Finally, the tetanus toxin and the botulinum toxins resemble each other in both biosynthesis and molecular architecture. Thus, there is an overall 34% identity between the protein sequences of tetanus toxin and botulinum toxin type A, and a sequence identity as high as 62% for some functional domains. Binz T. et al., The Complete Sequence of Botulinum Clostridial toxin Type A and Comparison with Other Clostridial toxins, J Biological Chemistry 265(16); 9153-9158: 1990.

### Acetylcholine

Typically only a single type of small molecule neurotransmitter is released by each type of neuron in the mammalian nervous system. The neurotransmitter acetylcholine is secreted by neurons in many areas of the brain, but specifically by the large pyramidal cells of the motor cortex, by several different neurons in the basal ganglia, by the motor neurons that innervate the skeletal muscles, by the preganglionic neurons of the autonomic nervous system (both sympathetic and parasympathetic), by the postganglionic neurons of the parasympathetic nervous system, and by some of the postganglionic neurons of the sympathetic nervous system. Essentially, only the postganglionic sympathetic nerve fibers to the sweat glands, the piloerector muscles and a few blood vessels are cholinergic as most of the postganglionic neurons of the sympathetic nervous system secret the neurotransmitter norepinephrine. In most instances acetylcholine has an excitatory effect. However, acetylcholine is known to have inhibitory effects at some of the peripheral parasympathetic nerve endings, such as inhibition of heart rate by the vagal nerve.

The efferent signals of the autonomic nervous system are transmitted to the body through either the sympathetic nervous system or the parasympathetic nervous system. The preganglionic neurons of the sympathetic nervous system extend from preganglionic sympathetic neuron cell bodies located in the intermediolateral horn of the spinal cord. The preganglionic sympathetic nerve fibers, extending from the cell body, synapse with postganglionic neurons located in either a paravertebral sympathetic ganglion or in a prevertebral ganglion. Since the preganglionic neurons of both the sympathetic and parasympathetic nervous system are cholinergic, application of acetylcholine to the ganglia will excite both sympathetic and parasympathetic postganglionic neurons.

Acetylcholine activates two types of receptors, muscarinic and nicotinic receptors. The muscarinic receptors are found in all effector cells stimulated by the postganglionic, neurons of the parasympathetic nervous system as well as in those stimulated by the postganglionic cholinergic neurons of the sympathetic nervous system. The nicotinic receptors are found in the adrenal medulla, as well as within the autonomic ganglia, that is on the cell surface of the postganglionic neuron at the synapse between the preganglionic and postganglionic neurons of both the sympathetic and parasympathetic systems. Nicotinic receptors are also found in many nonautonomic nerve endings, for example in the membranes of skeletal muscle fibers at the neuromuscular junction.

Acetylcholine is released from cholinergic neurons when small, clear, intracellular vesicles fuse with the presynaptic neuronal cell membrane. A wide variety of non-neuronal secretory cells, such as, adrenal medulla (as well as the PC12 cell line) and pancreatic islet cells release catecholamines and parathyroid hormone, respectively, from large dense-core vesicles. The PC12 cell line is a clone of rat pheochromocytoma cells extensively used as a tissue culture model for studies of sympathoadrenal development. Botulinum toxin inhibits the release of both types of compounds from both types of cells in vitro, permeabilized (as by electroporation) or by direct injection of the toxin into the denervated cell. Botulinum toxin is also known to block release of the neurotransmitter glutamate from cortical synaptosomes cell cultures.

A neuromuscular junction is formed in skeletal muscle by the proximity of axons to muscle cells. A signal transmitted through the nervous system results in an action potential at the terminal axon, with activation of ion channels and resulting release of the neurotransmitter acetylcholine from intraneuronal synaptic vesicles, for example at the motor endplate of the neuromuscular junction. The acetylcholine crosses the extracellular space to bind with acetylcholine receptor proteins on the surface of the muscle end plate. Once sufficient binding has occurred, an action potential of the muscle cell causes specific membrane ion channel changes, resulting in muscle cell contraction. The acetylcholine is then released from the muscle cells and metabolized by cholinesterases in the extracellular space. The metabolites are recycled back into the terminal axon for reprocessing into further acetylcholine.

As mentioned, there can be unpleasant effects associated with various bariatric procedures, those unpleasant side effects including nausea or vomiting. In addition, certain bariatric procedures may be more difficult to perform when the surgeon is required to work with the un-relaxed stomach muscles. What is also needed is an improved method of working with the stomach muscles whereby the stomach muscles are relaxed and thereby easier to work with.

What is needed, therefore, is an improved method of performing bariatric procedures that avoids the unwanted side effects of nausea and vomiting.

### SUMMARY OF THE INVENTION

The present invention addresses the above-described problems by using botulinum toxin prior to or during procedures where the physiology of the stomach is altered or an object is inserted in the stomach. The method of the present invention is discussed in the context of several different types of bariatric procedures, however the methods disclosed may be employed in any procedure where the physiology of the stomach is altered such that the patient experiences unwanted side effects of nausea and vomiting.

More particularly, botulinum toxin would be delivered via either an endoscopic (sometimes referred to herein as gastroscopic) and/or laparoscopic procedure prior to the insertion of the intragastric balloon or intragastric band, application of a gastric band or performing a gastric bypass procedure. While the present invention is discussed in the context of bariatric procedures, including the implantation of intragastric balloons and band, gastric bands, and gastric bypass procedures, it should be understood by those skilled in the art that the botulinum toxin may be used in any procedure where the physiology of the stomach is altered or an object is inserted in or on the stomach. Bariatric procedures are used as examples for the preferred embodiments of the present invention, as such procedures often have unwanted side effects associated with them, including pain, nausea, and vomiting. The physician may use neurotoxins when the physician wishes to lessen discomfort and pain and lessen unwanted side effects of vomiting and nausea in any procedure where the physiology of the stomach is altered.

Pretreatment with the botulinum toxin will lessen the feeling of a foreign body sensation and provide improved patient acceptance and outcomes for bariatric procedures. In addition, the use of a botulinum toxin can provide additional benefits in weight loss management by providing incremental reductions of BMI during the 6 month treatment timeframe. Botulinum toxin also improves the outcome of the procedure by relaxing the stomach muscles, thus making it easier to insert, remove, or replace an object, such as a gastric band or intragastric balloon in or around the stomach at any time over the treatment period.

In some embodiments, the methods comprise the steps of administering a neurotoxin to a stomach tissue of a patient and deploying a device such as an intragastric balloon or gastric band in or around the stomach of the patient, or performing a gastric bypass procedure. The neurotoxin (e.g., botulinum toxin types A, B, C₁, D, E, F and G) may be locally administered or orally administered. In some embodiments, the neurotoxin is locally administered at or in a vicinity of the site where the device is to be implanted or the gastric bypass is to be performed.

In some embodiments, the neurotoxin is administered to a stomach tissue prior to the step of deploying a device such as an intragastric balloon or gastric band in or around the stomach of the patient, or performing a gastric bypass procedure. One of the advantages in pre-administering the stomach with a neurotoxin is that it relaxes the stomach and makes it more malleable. When the stomach is relaxed and is more malleable, it is easier for the surgeon to maneuver the device in or on the stomach or perform the bypass procedure, which may result in reduced operation time and faster recovery.

In some embodiments, the neurotoxin is administered at or in the vicinity of the site where a device such as an intragastric balloon or gastric band contacts the stomach. This particular method is particularly advantageous for the implantation of a gastric band around the stomach, because the local administration of a neurotoxin at a site or in the vicinity of the site where the gastric band contacts the stomach relaxes the muscle in that particular region and allows the gastric band to stay located at that sight. Without wishing to limit the invention to any theory or mechanism of operation, it is believed that the administration of the neurotoxin at or in the vicinity of the site where the band contacts the stomach creates a contrast region in muscle tone that would serve to allow the band to settle in place. For example, when the neurotoxin is administered at the site where the band contacts the stomach, the site administered has a relaxed muscle tone. The gastric band would tend to “fall” into the region with the relaxed muscle tone—thus, the band would be secured in its intended site. In some embodiments, the gastric band is secured in that it does not twist around the stomach. In some embodiments, the gastric band is secured in that it does not slip off from the stomach.

Alternatively, the neurotoxin may be administered in the vicinity of the site where the stomach contacts the gastric band to create a contrast muscle tone region that would serve to hold the band in place. For example, a neurotoxin may be administered at a site above and/or below the site where the gastric band contacts the stomach (see FIGS. 3A and 3B). This pattern of administration would create a contrast in muscle tone region such that the gastric band would tend to “fall” into the region that is not administered.

The term “neurotoxin” employed herein refers to one or more of a toxin made by a bacterium, for example, a Clostridium botulinum, Clostridium butyricum, Clostridium beratti, Clostridium tetani. In some embodiments, the neurotoxin is a botulinum toxin. The botulinum toxin may be a botulinum toxin type A, type B, type C₁, type D, type E, type F, or type G. In some embodiments, the neurotoxin is a botulinum toxin type A. Unless stated otherwise, the dose of the neurotoxin referenced herein is equivalent to that of a botulinum toxin type A. The assays required to determine equivalency to the therapeutic effectiveness of botulinum toxin type A at a certain dosage are well established.

Further, the botulinum toxin of the present invention may comprise a first element comprising a binding element able to specifically bind to a neuronal cell surface receptor under physiological conditions, a second element comprising a translocation element able to facilitate the transfer of a polypeptide across a neuronal cell membrane, and a third element comprising a therapeutic element able, when present in the cytoplasm of a neuron, to inhibit exocytosis of acetylcholine from the neuron. The therapeutic element can cleave a SNARE protein, thereby inhibiting the exocytosis of acetylcholine from the neuron. The SNARE protein can be selected from the group consisting of syntaxin, SNAP-25 and VAMP.

### DEFINITIONS

The following definitions apply herein.

“About” means plus or minus ten percent of the value so qualified.

“Biocompatible” means that there is an insignificant inflammatory response upon ingestion of an oral formulation of a Clostridial toxin, as set forth herein.

“Effective amount” as applied to the biologically active compound means that amount of the compound which is generally sufficient to effect a desired change in the subject.

“Effective amount” as applied to a non-active ingredient constituent of an oral formulation (such as a polymer used for forming a matrix or a coating composition) refers to that amount of the non-active ingredient constituent which is sufficient to positively influence the release of a biologically active agent at a desired rate for a desired period of time. For example, where the desired effect is muscle paralysis by using a single oral formulation, the “effective amount” is the amount that can facilitate extending the release over a period of between about 60 days and 6 years. This “effective amount” can be determined based on the teaching in this specification and the general knowledge in the art.

“Effective amount” as applied to the amount of surface area of an oral formulation is that amount of oral formulation surface area which is sufficient to effect a flux of biologically active compound so as to achieve a desired effect, such as a muscle paralysis or a decrease in the secretory activity of a gland. The area necessary may be determined and adjusted directly by measuring the release obtained for the particular active compound. The surface area of the oral formulation or of a coating of an oral formulation is that amount of membrane necessary to completely encapsulate the biologically active compound. The surface area depends on the geometry of the oral formulation. Preferably, the surface area is minimized where possible, to reduce the size of the oral formulation.

“Locally administering” or “local administration” means direct injection of a tissue, e.g., stomach tissue. For example, local administration to a stomach tissue may be accomplished by using an endoscope and a sclerotherapy needle (see U.S. Pat. No. 5,437,291, the disclosure of which is incorporated in its entirety herein by reference). Alternatively, a gastroscopic instrument may be used to administer the neurotoxin.

“Oral formulation” means a drug delivery system intended for oral ingestion. The oral formulation can be comprised of a biocompatible polymer or natural material which contains or which can act as a carrier for a molecule with a biological activity.

“Deploying” an intragastric balloon in the stomach means inserting the balloon in the stomach and positioning it at a desirable location.

“Deploying” a gastric band around the stomach means wrapping the band around the stomach and positioning it at a desirable location, so that when tightened, the band pinches the stomach into an upper and a lower portion.

“Gastric bypass” means the surgical procedure for creating a small pouch in the stomach for the digestion of food that bypasses the remainder of the stomach.

“Treatment” means any treatment of a disease (obesity) in a mammal, and includes: (i) preventing the disease from occurring or; (ii) inhibiting the disease, i.e., arresting its development; (iii) relieving the disease, i.e., reducing the incidence of symptoms of or causing regression of the disease.

### BRIEF DESCRIPTION OF THE DRAWINGS

FIGS. 1A, 1B, and 1C show the general diagram of the stomach; the longitudinal and circular muscular fibers of the stomach, viewed from above and in front; and the oblique muscular fibers of the stomach, viewed from above and in front, respectively.

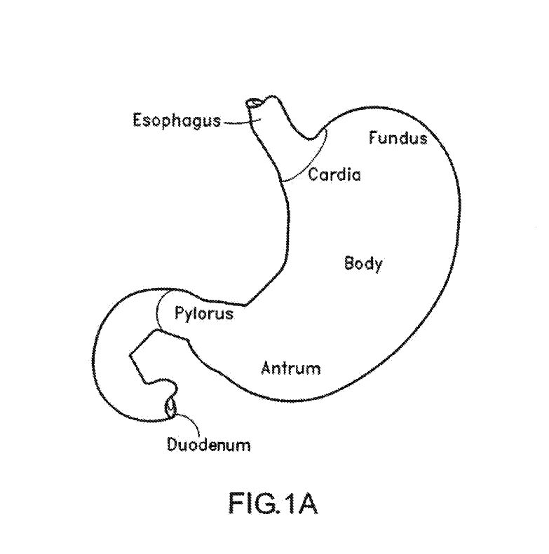

FIGS. 2A and 2B show examples of an administration of a neurotoxin in the vicinity of the site where an intragastric balloon contacts the stomach, and more generalized injection sites throughout the stomach.

FIGS. 3A and 3B show examples of an administration of a neurotoxin at a site where the gastric band contacts the stomach, and in the vicinity of the site where the gastric band contacts the stomach.

FIGS. 4A and 4B show examples of administration of a neurotoxin at a site where a gastric bypass is to be performed, and in the vicinity of the site where gastric bypass is to be performed.

FIG. 5 shows the insertion of a modified flexible endoscope through the esophagus for administration of a neurotoxin in interior of the stomach.

FIG. 6 show the insertion of a laparoscope for the administration of a neurotoxin on the exterior of the stomach.

FIG. 7 shows an intragastric balloon fully inserted in the stomach after a neurotoxin has been administered to the stomach.

FIG. 8 shows a gastric band in position around the stomach after a neurotoxin has been administered to the stomach.

FIG. 9 shows a stomach after a Roux en-Y Proximal gastric bypass procedure has been performed after a neurotoxin has been administered in the stomach.

### DETAILED DESCRIPTION OF THE PREFERRED EMBODIMENTS

Under the method of the present invention, various surgical procedures are performed on the stomach, including insertion of gastric bands and intragastric balloons, gastric bypass, and any other procedures where the physiology of the stomach is altered or an object is inserted in or on the stomach. The methods disclosed utilize an administration of a neurotoxin, such as a botulinum toxin, to a stomach tissue that allows the stomach muscles to be relaxed and therefore more easily maneuvered to facilitate performance of bariatric procedures. The administration of a neurotoxin in the stomach lessens the feeling of a foreign body sensation in the stomach and provides improved patient acceptance and outcomes for intragastric balloons. This significantly reduces or in many cases eliminates the unwanted side effects, including nausea and vomiting.

In some embodiments, the methods comprise the steps of administering a neurotoxin to a stomach tissue of a patient, and deploying an intragastric balloon in the stomach of the patient. In some embodiments, the methods comprise the steps of administering a neurotoxin to a stomach tissue of a patient, and deploying a gastric band around the stomach. In some embodiments, the methods comprise the steps of administering a neurotoxin to a stomach tissue of a patient, and performing a gastric bypass procedure. In some embodiments, the stomach tissue is a smooth muscle of the stomach, e.g., longitudinal, circular and/or oblique. In some embodiments, the neurotoxin is administered to the circular muscle of the stomach.

The neurotoxin (e.g., botulinum toxin types A, B, C₁, D, E, F and G) may be locally administered. The neurotoxin may be locally administered using an endoscopic and/or laparoscopic procedure (see Example 2 below). In some embodiments, the neurotoxin is administered generally around the area where a device, such as an intragastric balloon or gastric band is to be deployed, or where a surgical procedure, such as a gastric bypass, is to be performed. In some embodiments, the neurotoxin is administered to a stomach tissue prior to the step of deploying a device or performing another bariatric surgical procedure.

Various references have disclosed an endoscopic administration of botulinum toxin to a stomach to treat obesity. See, for example, Porta et al. (Mov Disord 2004, 19(9):S431 ABP1264); Albani et al. (J. Gastroenterol 2005, 40:833-835); Garcia-Compean et al. (Gastroenterol Clin Biol 2005, 29(8-9):789-791); U.S. Pat. App. Pub. 20040009224 to Miller; and U.S. Pat. App. Pub. 20040037865. These references disclose that the administration of a botulinum toxin to the stomach is effective to reduce motility of the stomach muscle (to slow down stomach emptying) and/or reduce the secretion of ghrelin, which presents a powerful signal of “hunger sensation” to the hypothalamus. However, the references do not teach or suggest that an administration of a neurotoxin, such as botulinum, can be used in conjunction with the insertion of a device such as a gastric band or intragastric balloon, or the performance of a surgical procedure, such as gastric bypass. More specifically, these references do not teach or suggest that administration of a neurotoxin to a stomach tissue allows an intragastric balloon or gastric band to be more easily maneuvered in the stomach and subsequently adjusted, or that an administration of a neurotoxin at or in the vicinity of a surgical procedure makes the stomach muscle more malleable and relaxed at the site of the procedure. These references also do not teach that the administration of a neurotoxin in conjunction with a procedure wherein the physiology of the stomach is altered significantly minimizes or eliminates unwanted side effects such as pain, nausea, or vomiting.

In some embodiments, the neurotoxin is orally administered. The neurotoxin may be administered to the stomach via an oral ingestion of a neurotoxin oral formulation. For example, a neurotoxin oral formulation within the scope of the present invention is capable of releasing an effective amount of a neurotoxin into the stomach of a patient to relax the stomach muscle. The amount of released neurotoxin can comprise as little as about 10 units (based on the units of botulinum toxin type A) (i.e. to relax the stomach muscle of a patient weighing less than 50 kg) to as much as 500 units (i.e. to relax the stomach muscle of a large adult). The quantity of botulinum toxin required to effectively relax a stomach muscle can be varied according to the known clinical potency of the different neurotoxins, e.g., botulinum toxin serotypes. For example, several orders of magnitude more units of a botulinum toxin type B are typically required to achieve a physiological effect comparable to that achieved from use of a botulinum toxin type A.

The specific dosage by oral formulation appropriate for administration is readily determined by one of ordinary skill in the art according to the factors discussed above. The dosage can also depend upon the size of the tissue mass to be treated or denervated, and the commercial preparation of the toxin. Additionally, the estimates for appropriate dosages in humans can be extrapolated from determinations of the amounts of botulinum required for effective denervation of other tissues. Thus, the amount of botulinum A to be injected is proportional to the mass and level of activity of the tissue to be treated. Generally, between about 0.01 units per kilogram to about 35 units per kg of patient weight of a botulinum toxin, such as botulinum toxin type A, can be released by the present oral formulation per unit time period (i.e. over a period of or once every 2-4 months) to effectively accomplish a desired relaxation of the stomach muscle. Less than about 0.01 U/kg of a botulinum toxin may not have a significant therapeutic effect upon a stomach endocrine cell, while more than about 35 U/kg of a botulinum toxin approaches a toxic dose of a Clostridial toxin, such as a botulinum toxin type A. Careful preparation of the oral formulation prevents significant amounts of a botulinum toxin from appearing systemically. A more preferred dose range is from about 0.01 U/kg to about 25 U/kg of a botulinum toxin, such as that formulated as BOTOX®. The actual amount of U/kg of a botulinum toxin to be administered depends upon factors such as the extent (mass) and level of activity of the tissue to be treated and the administration route chosen. Botulinum toxin type A is a preferred botulinum toxin serotype for use in the methods of the present invention.

The oral formulation may be prepared so that the neurotoxin is substantially uniformly dispersed in a biodegradable carrier. An alternate oral formulation within the scope of the present invention can comprise a carrier coated by a biodegradable coating, either the thickness of the coating or the coating material being varied.

The thickness of the oral formulation can be used to control the absorption of water by, and thus the rate of release of a neurotoxin from, a composition of the invention, thicker oral formulations releasing the polypeptide neurotoxin more slowly than thinner ones.

The neurotoxin in a neurotoxin controlled release composition can also be mixed with other excipients, such as bulking agents or additional stabilizing agents, such as buffers to stabilize the neurotoxin during lyophilization. Additional details regarding a neurotoxin formulation suitable for oral delivery may be found in, for example, U.S. Patent Publication 20040086532 and U.S. Patent Publication 20040253274, the disclosures of which are incorporated in their entirety herein by reference.

In some embodiments, the neurotoxin is administered to the stomach prior to deploying a device such as an intragastric balloon or gastric band in or on the stomach, or before the performance of a surgical procedure, such as a gastric bypass. One of the advantages in pre-administering the stomach with a neurotoxin is that it relaxes the stomach and makes it more malleable. When the stomach is relaxed and is more malleable, it is easier for the surgeon to maneuver in and around the stomach, which would result in reduced operation time and faster recovery. For example, a standard gastric band procedure takes about 30-45 minutes. With a pre-administration of a neurotoxin, the procedure may be faster by about 10-40%, as the surgeon is better able to maneuver around a more malleable stomach. Also, a pre-administration of a neurotoxin results in a faster healing time. For example, after a conventional gastric band procedure, most patients are able to return to normal functions after about 5-7 days. However, an administration of a neurotoxin prior to a gastric band procedure may result in patients being able to return to normal functions about 10-40% faster, as compared to patients undergoing the same procedure but without the pre-administration of a neurotoxin. The patients will also feel much better, as the unwanted side effects of nausea and vomiting are drastically reduced, and in many instances completely eliminated.

Another advantage of administering the neurotoxin to a stomach tissue prior to deploying a device in the stomach, such as a gastric band or intragastric balloon, or performing a surgical procedure in the stomach, such as gastric bypass, is that the stomach is relaxed and it is easier to make adjustments to the device or at the procedure site. For example, after the intragastric balloon is deployed in the stomach, the patient is scheduled for a regular check up. During this check up, the balloon may be adjusted to decrease or increase the size of the balloon. This is a quick and relatively painless outpatient procedure. The balloon may be adjusted using a gastroscopic instrument, such as described in commonly assigned U.S. patent application Ser. No. 11/540,177. Depending on the patient's needs, the surgeon may wish to add or remove saline from the balloon. Adding saline increases the size of balloon, further restricting the amount of food the patient can eat before feeling full and satisfied. When the stomach is administered with a neurotoxin, the stomach muscles are more malleable, facilitating the adjustment and manipulation of the balloon in situ. In addition, when the stomach is administered a neurotoxin, unwanted side effects, such as pain, discomfort, nausea and vomiting, that may accompany the adjustment procedure, are greatly reduced and oftentimes eliminated.

The invention also includes a method for facilitating weight loss by deploying a device, such as an intragastric balloon or gastric band, in the stomach of the patient wherein the device has previously been coated with a botulinum toxin, such as botulinum toxin type A, on the surface that will be in contact with stomach tissue. Thus, when the intragastric balloon comes in contact with the stomach, the botulinum toxin is absorbed into or diffuses into the adjacent stomach tissue. Technologies for coating a medical device with a botulinum toxin are known. See, e.g. U.S. Pat. Nos. 6,767,544 and 6,579,847.

In some embodiments, the neurotoxin is administered at or in the vicinity of the site where a device to be implanted, such as a gastric band or intragastric balloon, contacts the stomach. Particularly with respect to gastric band applications, one of the advantages of locally administering a neurotoxin at a site or in the vicinity of the site where a device contacts the stomach is that the band is better fitted at that site and does not tend to slip from that site. Without wishing to limit the invention to any theory or mechanism of operation, it is believed that the administration of the neurotoxin at or in the vicinity of the site where the band contacts the stomach creates a contrast in muscle tone region that would serve to create a resting place for the intragastric balloon. For example, when the neurotoxin is administered at the site where the band contacts the stomach, the site administered has a relaxed muscle tone. The gastric band would tend to “fall” into the region with the relaxed muscle tone—thus, the band would rest in its intended location. One or more sites on the stomach may be administered with neurotoxin. In some embodiments, the neurotoxin is administered along the entire circumference of the stomach. In some embodiments, the neurotoxin is administered substantially on the greater curvature side of the stomach. In some embodiments, the neurotoxin is administered on the stomach at sites that are about 1-10 cm apart. In some embodiments, about 0.5-10 units (based on botulinum toxin type A) of a neurotoxin are administered to each site.

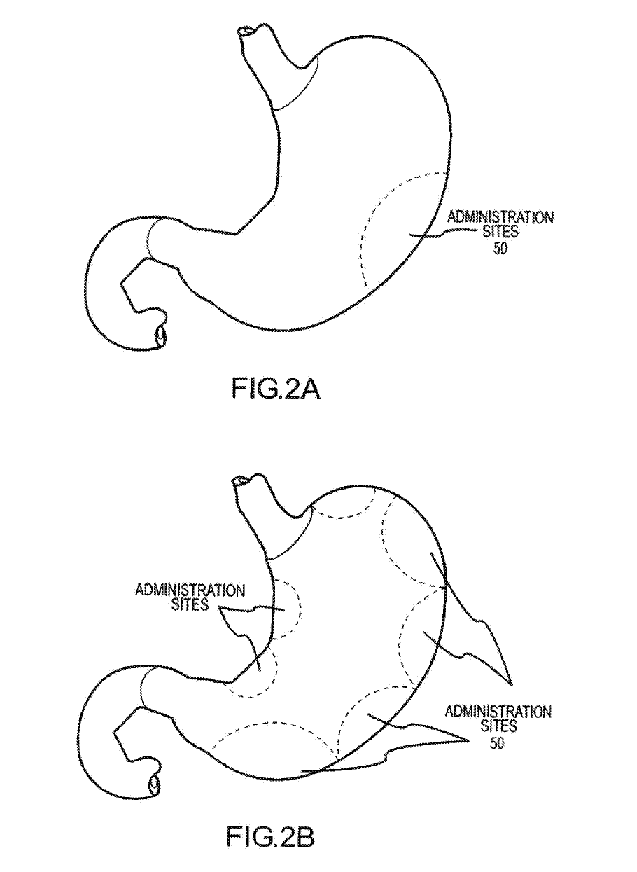

Alternatively, the neurotoxin may be administered in the vicinity of the site where the stomach contacts the gastric band to create a contrast muscle tone region that would serve to secure the band in place. For example, a neurotoxin may be administered at a site above and/or below the site where the gastric band contacts the stomach (see FIGS. 3A and 3B). This pattern of administration would create a contrast in muscle tone such that the gastric band would tend to “fall” into the region that is not administered. In some embodiments, the neurotoxin is administered along the entire circumference of the stomach. In some embodiments, the neurotoxin is administered substantially on the greater curvature side of the stomach. In some embodiments, the neurotoxin is administered on the stomach at sites that are about 1-10 cm apart. In some embodiments, about 0.5-10 units (based on botulinum toxin type A) of a neurotoxin are administered to each site.

Preferably, a neurotoxin used to practice a method within the scope of the present invention is a botulinum toxin, such as one of the serotype A, B, C, D, E, F or G botulinum toxins. More preferably, the botulinum toxin used is botulinum toxin type A, because of its high potency in humans, ready availability, and known safe and efficacious use for the treatment of skeletal muscle and smooth muscle disorders when locally administered by intramuscular injection.

The present invention includes within its scope: (a) Clostridial toxin complex as well as pure Clostridial toxin obtained or processed by bacterial culturing, toxin extraction, concentration, preservation, freeze drying and/or reconstitution, and (b) modified or recombinant Clostridial toxin, that is Clostridial toxin that has had one or more amino acids or amino acid sequences deliberately deleted, modified or redeployed by known chemical/biochemical amino acid modification procedures or by use of known host cell/recombinant vector recombinant technologies, as well as derivatives or fragments of Clostridial toxins so made, and includes Clostridial toxins with one or more attached targeting moieties for a cell surface receptor present on a cell.

Neurotoxins, e.g., botulinum toxins, for use according to the present invention can be stored in lyophilized or vacuum dried form in containers under vacuum pressure. Prior to lyophilization the botulinum toxin can be combined with pharmaceutically acceptable excipients, stabilizers and/or carriers, such as albumin. The lyophilized or vacuum dried material can be reconstituted with saline or water.

Methods for determining the appropriate route of administration and dosage are generally determined on a case by case basis by the attending physician. Such determinations are routine to one of ordinary skill in the art (see for example, Harrison's Principles of Internal Medicine (1998), edited by Anthony Fauci et al., 14^{th} edition, published by McGraw Hill).

### EXAMPLES

The following examples, describing various procedures using the devices and methods of the present invention, are for illustrative purposes only and are not intended, nor should they be interpreted, to limit the scope of the invention.

### Example 1

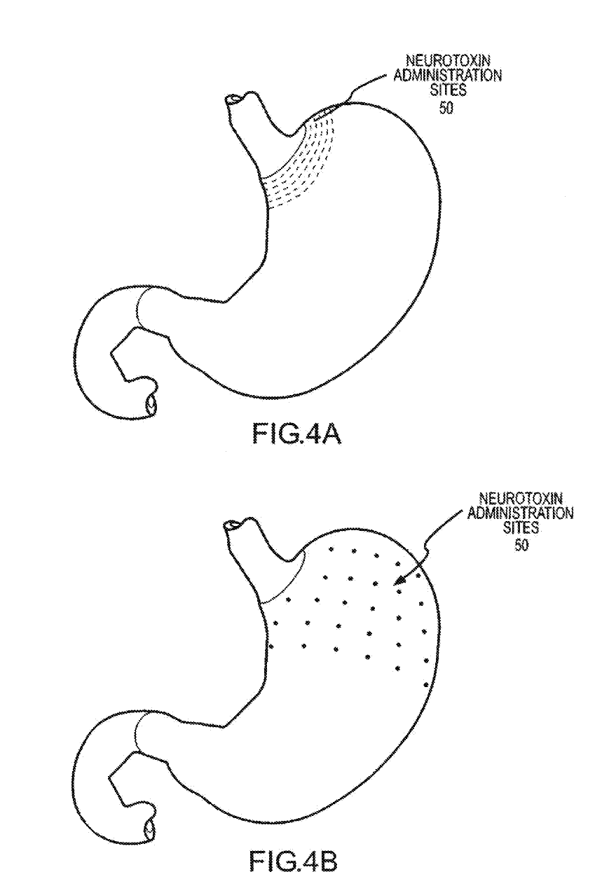

In this first example, an endoscopic procedure is described to locally inject botulinum toxin inside the stomach. Reference is made to FIG. 5.

A middle age male patient has a BMI (Body Mass Index) of between 30-40. The patient is a good candidate for an intragastric balloon procedure to help him lose weight.

The patient wishes to lose weight and elects to undergo a BioEnterics® Intragastric Balloon (BIB®) System, for example.

To locally administer a neurotoxin to a stomach site, an endoscopy is performed with a standard adult forward-viewing gastroscopic instrument. The site of administration on the stomach is estimated both gastroscopically as well as by a previously performed manometry. At the administration site, a neurotoxin, e.g., botulinum toxin type A, is injected via a 4-mm sclerotherapy needle passed thorough the biopsy channel of the gastroscope 24 (FIG. 5). One milliliter of a 10 U/mL solution can be injected into each site on the stomach (see U.S. Pat. No. 5,437,291, the disclosure of which is incorporated in its entirety herein by reference).

Once the neurotoxin has been administered to the stomach muscles, the intragastric balloon may be introduced into the stomach gastroscopically and inflated using conventional endoscopy techniques known to those skilled in the art.

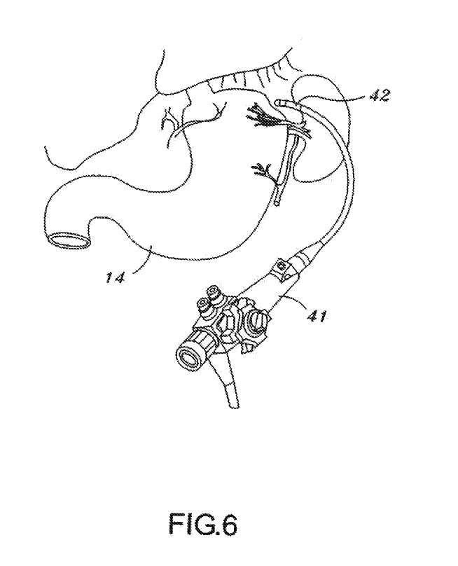

The BIB® procedure is performed after the surgeon determines that the stomach is adequately relaxed by the administration of a botulinum toxin. The recovery time from the BIB® procedure performed after the stomach is relaxed by the administration of a botulinum toxin is faster as compared to that of the same procedure where the stomach is not relaxed by the administration of a botulinum toxin. In a typical BIB placement, a patient experiences a vomiting reflex for about two days following the procedure, although in extreme cases, the reflex may last as many as five days after the procedure is performed. In the case where botulinum toxin has been administered prior to or during surgery, the recovery time is reduced significantly, and the vomiting reflex may be alleviated in as little as a day or less. FIG. 7 shows an intragastric balloon in place in the stomach with the patient having undergone the procedure described herein.

### Example 2

In this second example, a laparoscopic procedure is described to deploy a gastric band, with neurotoxin being administered prior to the implantation of the gastric band. Reference is made to FIGS. 3A, 3B, 6, and 8.

A middle age female patient has a BMI (Body Mass Index) of between 30-60. The patient is a good candidate for a gastric band procedure to help her lose weight.

The patient wishes to lose weight and elects to undergo a LAP-BAND® procedure, for example.

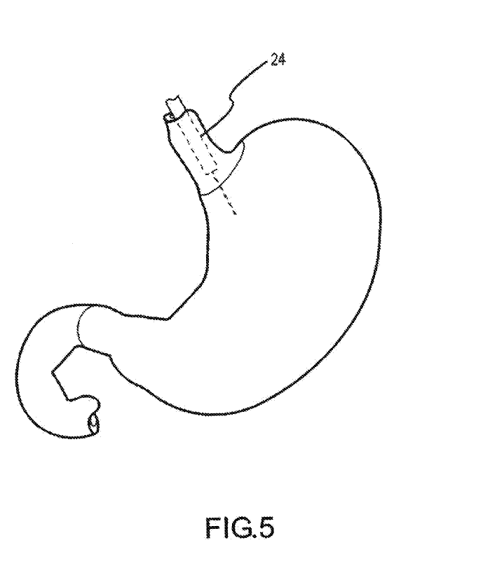

Routine procedures for laparoscopic surgical entrance into the abdominal cavity are followed, using surgical procedures known to those skilled in the art. A laparoscope 41 (FIG. 6) is used to view the stomach and perform the procedure in a minimally invasive procedure. The optical system of the laparoscope is useful in positioning the needle that is attached to the tip of the laparoscope for injection of neurotoxin, preferably botulinum toxin type A. Once the laparoscope is positioned at the appropriate injection sites, a needle equipped on the tip of the laparoscope may be used to locally administer an effective amount of neurotoxin, as is shown in FIG. 6. Alternatively, a needle may be separately introduced through a working channel of the laparoscope or a separate laparoscopic cannula.

As an alternative to laparoscopic injection, a gastroscope may be used to enter the stomach through the esophagus via the mouth to locally administer an effective amount of neurotoxin inside the stomach, as is shown in FIG. 5. Neurotoxin administration sites 50 are shown in FIGS. 3A and 3B.

Once the neurotoxin has been administered to the stomach muscles, the gastric band may be inserted around the stomach laparoscopically using surgical techniques known to those skilled in the art.

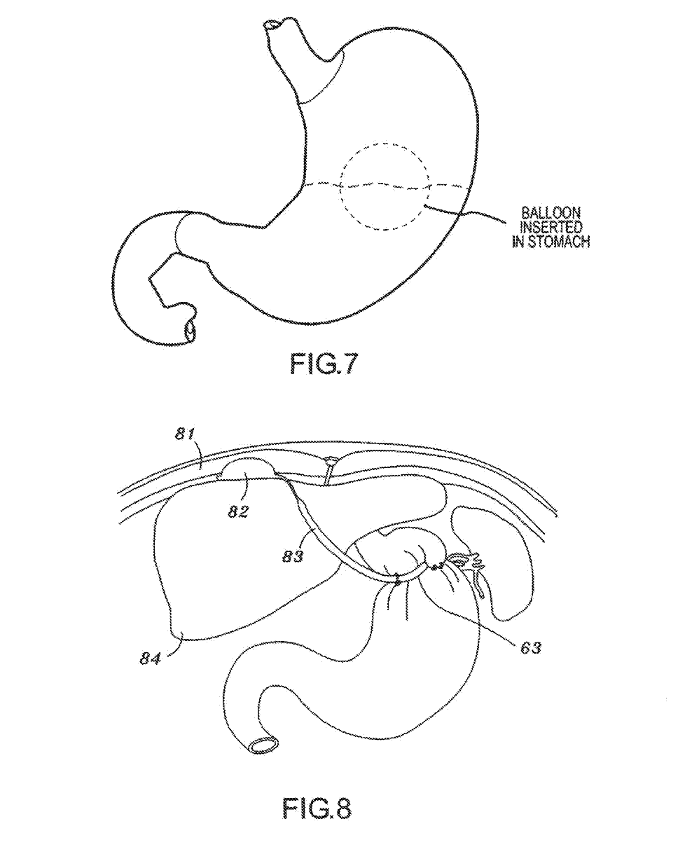

The LAP-BAND® procedure is performed after the surgeon determines that the stomach is adequately relaxed by the administration of a botulinum toxin. The LAP-BAND® procedure takes less time as compared to the same procedure where the stomach is not relaxed by the administration of a botulinum toxin, as the surgeon can maneuver around the stomach more easily. In this case, the LAP-BAND® procedure is around 25 minutes, which is about 5 minutes faster than usual. Moreover, the recovery time from the LAP-BAND® procedure performed after the stomach is relaxed by the administration of a botulinum toxin is faster as compared to that of the same procedure where the stomach is not relaxed by the administration of a botulinum toxin. In this case, the recovery time is about 3 days, which is about 2-3 days faster than usual. FIG. 8 shows a gastric band 21 in its place with the patient having undergone the procedure described herein.

### Example 3

In this third example, a method for facilitating weight loss with local administration of botulinum toxin to the stomach followed by implantation of a gastric band is discussed.

In this example, the patient is a male at least 60-100 pounds overweight. The patient is a good candidate for a gastric band procedure to help him lose weight.

The patient wishes to lose weight and elects to undergo a LAP-BAND® procedure. A few weeks prior to and/or at the time of the actual LAP-BAND® procedure, the patient is administered with a botulinum toxin to relax the stomach muscles. Using gastroscopic techniques, the botulinum toxin is administered to the upper part of the stomach, preferably to or in the vicinity of a site where the band is to be deployed (“in the vicinity” of the site means, for example, within about less than 10 cm from the site of where the band is to be deployed on the stomach).

The time gap between the pre-administration of the botulinum toxin and LAP-BAND® procedure depends on the dose and botulinum toxin type administered. Preferably, the muscle tone of the stomach muscle is relaxed by at least more than about 50% of the maximum contraction prior to performing LAP-BAND® procedure.

When the patient is ready for the LAP-BAND® procedure, the patient is placed on a no fat, liquid diet for 7 days before the surgery. The purpose of this liquid diet is to decrease the size of the liver, which in turn will make the placement of the LAP-BAND® safer.

The LAP-BAND® procedure performed after the stomach is relaxed by the administration of a botulinum toxin takes less time as compared to the same procedure where the stomach is not relaxed by the administration of a botulinum toxin, as the surgeon can maneuver around the stomach more easily. In this case, the LAP-BAND® procedure is around 25 minutes, which is about 10 minutes faster than usual. Moreover, the recovery time (time the patient is able to resume normal daily functions) from the LAP-BAND® procedure performed after the stomach is relaxed by the administration of a botulinum toxin is faster as compared to that of the same procedure where the stomach is not relaxed by the administration of a botulinum toxin. In this case, the recovery time is about 4 days, which is about 1 or 2 days faster than usual.

### Example 4

In this fourth example, a method for facilitating weight loss with oral administration of botulinum toxin to the stomach followed by the implantation of a gastric band is discussed.

A middle age female patient has a BMI (Body Mass Index) of between 30-60. The patient is a good candidate for a gastric band procedure to help her lose weight.

The patient wishes to lose weight and elects to undergo a LAP-BAND® procedure. A few weeks prior and/or at the time of the LAP-BAND® procedure, the patient is administered with an oral botulinum toxin formulation to relax the stomach muscles.

The time gap between the pre-administration of the botulinum toxin and LAP-BAND® procedure depends on the dose and botulinum toxin type administered. Preferably, the muscle tone of the stomach muscle is relaxed to at least more than about 75% of the maximum contraction prior to performing LAP-BAND® procedure.

The LAP-BAND® procedure is performed after the surgeon determines that the stomach is adequately relaxed by the administration of a botulinum toxin. The LAP-BAND® procedure takes less time as compared to the same procedure where the stomach is not relaxed by the administration of a botulinum toxin, as the surgeon can maneuver around the stomach more easily. In this case, the LAP-BAND® procedure is around 25 minutes, which is about 5 minutes faster than usual. Moreover, the recovery time from the LAP-BAND® procedure performed after the stomach is relaxed by the administration of a botulinum toxin is faster as compared to that of the same procedure where the stomach is not relaxed by the administration of a botulinum toxin. In this case, the recovery time is about 3 days, which is about 2-3 days faster than usual.

### Example 5

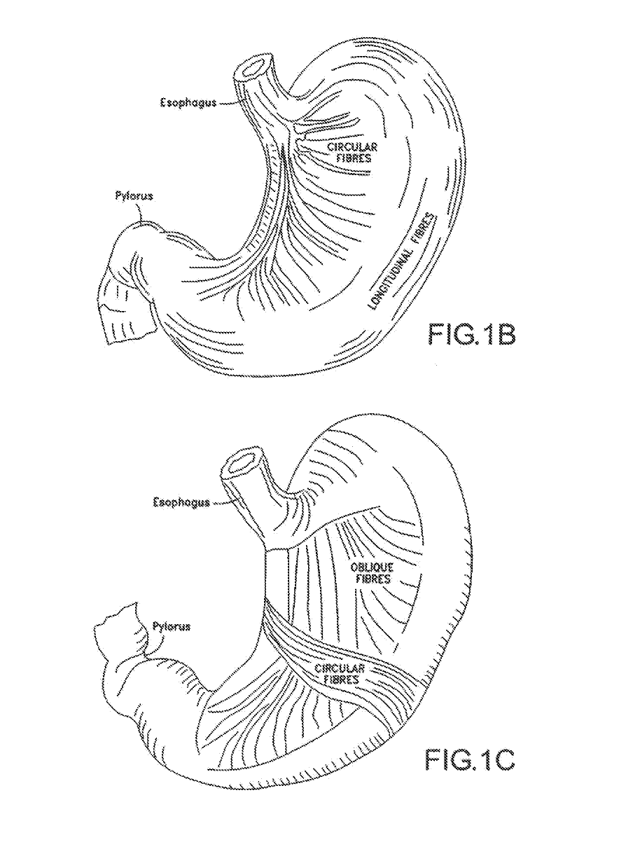

In this fifth example, a method for facilitating weight loss with administration of botulinum toxin to the stomach followed by the implantation of an intragastric balloon is discussed. Reference is made to FIGS. 2A, 2B, 5, and 7.

In this example, the patient wishes to lose weight and elects to undergo the balloon placement procedure, using an intragastric balloon such as the BioEnterics® Intragastric Balloon (BIB®) System.

A gastroscope 24 (FIG. 5) is used to enter the stomach through the esophagus via the mouth to conduct an initial examination of the stomach using an endoscopic camera. If no abnormalities are observed, the physician proceeds with the procedure. The physician uses either a needle attached to the end of the gastroscope or a needle that may be passed through the working channel of a gastroscope to locally administer an effective amount of neurotoxin, as is shown in FIG. 5. The neurotoxin is locally administered at administration sites 50, as shown in FIGS. 2A and 2B.

Once the neurotoxin has been administered to the stomach muscles, the intragastric balloon is introduced into the stomach gastroscopically using surgical techniques known to those skilled in the art.

The balloon is introduced into the stomach through the mouth without the need for surgery, with placement of the balloon through the mouth and down the esophagus and into the stomach. The balloon is made of a soft pliable silicone elastomer material and is inserted while in its smallest, deflated form. The swallowing process is made easier with the help of anesthetics applied topically to numb the throat area. Because the neurotoxin has been previously administered, the stomach muscles are relaxed, which facilitates introduction and filling of the balloon. Once the balloon is inside the stomach, it is immediately filled with sterile saline through a small filling tube, or catheter, attached to the balloon.

Once filled, the doctor removes the filling tube by gently pulling on the external end. The balloon has a self-sealing valve, and at this point the balloon is floating freely in the stomach. Placement times vary, but the procedure usually takes 30-60 minutes, after which the patient will be monitored by the physician for a short time and then may return home.

FIG. 7 shows an intragastric balloon fully inserted in the stomach according to the procedure described above.

### Example 6

In this sixth example, a method for facilitating weight loss with administration of botulinum toxin to the stomach followed by the implantation of an intragastric balloon is discussed. Reference is made to FIGS. 2A, 2B, 5, and 7.

In this example, the patient wishes to lose weight and elects to undergo the balloon placement procedure, using an intragastric balloon such as the BioEnterics® Intragastric Balloon (BIB®) System.

A few weeks prior to and/or at the time of the actual balloon placement procedure, the patient is administered with a botulinum toxin to relax the stomach muscles. Using gastroscopic techniques, the botulinum toxin is administered to the stomach. Reference is made to FIGS. 2A and 2B which show the various administration sites 50 in the stomach.

The time gap between the pre-administration of the botulinum toxin and the implantation of the intragastric balloon depends on the dose and botulinum toxin type administered.

At the time the balloon placement is to be performed, a gastroscope 24 (FIG. 5) is used to locally inject an additional amount of neurotoxin, if needed.

Once the neurotoxin has been administered, or if no additional neurotoxin is needed, the balloon is introduced into the stomach through the mouth without the need for surgery, with placement of the balloon through the mouth and down the esophagus into the stomach. The balloon is made of a soft pliable silicone elastomer material and is inserted while in its smallest, deflated form. The swallowing process is made easier with the help of anesthetics applied topically to numb the throat area. Because the neurotoxin has been previously administered, the stomach muscles are relaxed, which facilitates the balloon placement. Once the balloon is inside the stomach, it is immediately filled with sterile saline through a small filling tube, or catheter, attached to the balloon.

Once filled, the doctor removes the filling tube by gently pulling on the external end. The balloon has a self-sealing valve, and at this point the balloon is floating freely in the stomach. Placement times vary, but the procedure usually takes 30-60 minutes, after which the patient will be monitored by the physician for a short time and then may return home.

### Example 7

In this seventh example, a method for facilitating weight loss with oral administration of botulinum toxin to the stomach followed by the implantation of an intragastric balloon is discussed.

In this example, the patient wishes to lose weight and elects to undergo the balloon placement procedure, using an intragastric balloon such as the BioEnterics® Intragastric Balloon (BIB®) System.

A few weeks prior to and/or at the time of the actual balloon placement procedure, the patient is administered with an oral botulinum toxin formulation to relax the stomach muscles.

The time gap between the pre-administration of the botulinum toxin and the implantation of the gastric balloon depends on the dose and botulinum toxin type administered.

When the surgeon is ready to perform the placement of the balloon, the balloon is introduced into the stomach through the mouth without the need for surgery, with placement of the balloon through the mouth and down the esophagus and into the stomach. The balloon is made of a soft pliable silicone elastomer material and is inserted while in its smallest, deflated form. The swallowing process is made easier with the help of anesthetics applied topically to numb the throat area. Because the neurotoxin has been previously administered, the stomach muscles are relaxed, which facilitates the balloon placement. Once the balloon is inside the stomach, it is immediately filled with sterile saline through a small filling tube, or catheter, attached to the balloon.

Once filled, the doctor removes the filling tube by gently pulling on the external end. The balloon has a self-sealing valve, and at this point the balloon is floating freely in the stomach. Placement times vary, but the procedure usually takes 30-60 minutes, after which the patient will be monitored by the physician for a short time and then may return home. FIG. 7 shows an intragastric balloon fully inserted in the stomach according to the procedure described above.

### Example 8

In this eighth example, a method for facilitating weight loss with administration of botulinum toxin to the stomach followed by the performance of a gastric bypass procedure is discussed.

As can be understood from the examples discussed above, there are several different methods for administering a neurotoxin in conjunction with the performance of a bariatric procedure. In this example, botulinum toxin is administered orally prior to the performance of the gastric bypass procedure. However, as is understood from the above examples, the botulinum toxin may be injected prior to or during the performance of the gastric bypass procedure. In addition, the botulinum toxin may be administered using any combination of the methods discussed above, both before and during the performance of the procedure.

The patient wishes to lose weight and elects to undergo a gastric bypass procedure. A few weeks prior and/or at the time of gastric bypass procedure, the patient is administered with an oral botulinum toxin formulation to relax the stomach muscles.

The time gap between the pre-administration of the botulinum toxin and the gastric bypass procedure depends on the dose and botulinum toxin type administered. Preferably, the muscle tone of the stomach muscle is relaxed to at least more than about 75% of the maximum contraction prior to performing gastric bypass procedure.

The gastric bypass procedure is performed after the surgeon determines that the stomach is adequately relaxed by the administration of a botulinum toxin. The gastric bypass procedure takes less time as compared to the same procedure where the stomach is not relaxed by the administration of a botulinum toxin, as the surgeon can manipulate the stomach more easily. This allows the physician to perform the procedure more quickly. Moreover, the recovery time from the gastric bypass procedure performed after the stomach is relaxed by the administration of a botulinum toxin is faster as compared to that of the same procedure where the stomach is not relaxed by the administration of a botulinum toxin.

### Example 9

In this ninth example, a method for facilitating weight loss with administration of botulinum toxin to the stomach followed by the performance of a gastric bypass procedure is discussed.

As can be understood from the examples discussed above, there are several different methods for administering a neurotoxin in conjunction with the performance of a bariatric procedure. In this example, botulinum toxin is injected during the performance of the gastric bypass procedure.

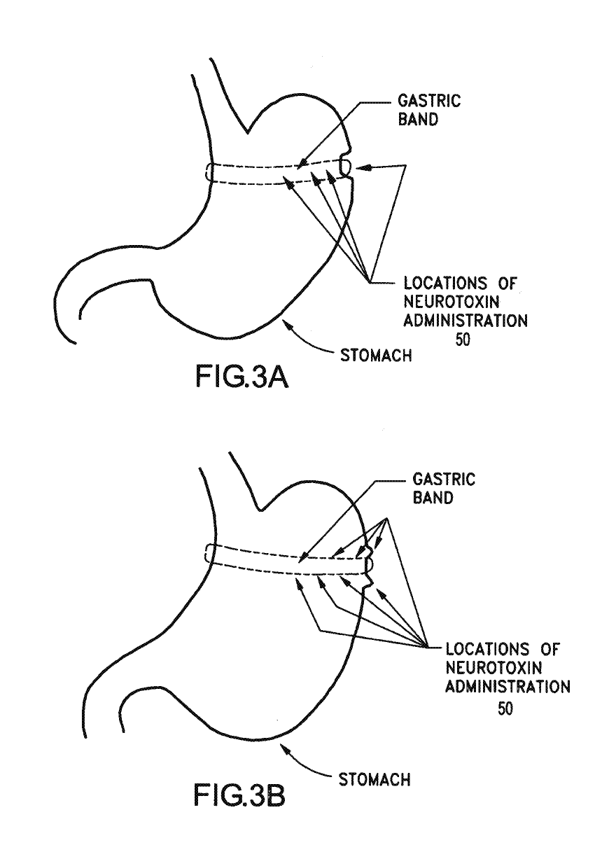

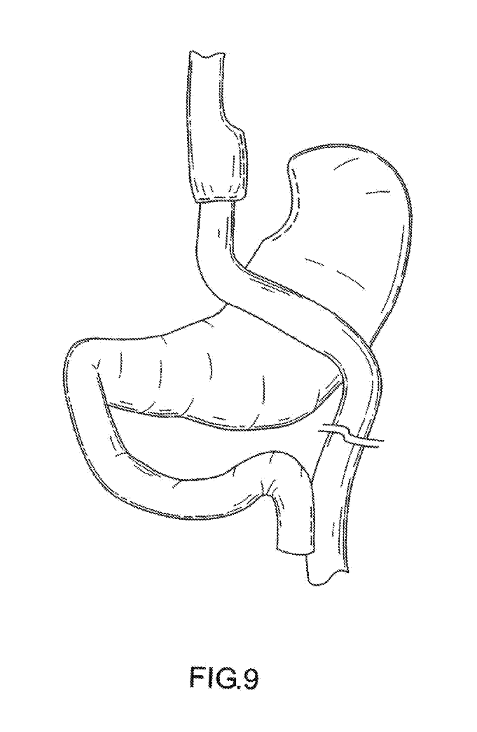

The physician gains access to the area of the stomach where the bypass procedure is to be performed, using methods known to those skilled in the art. In this example, a Roux en-Y Proximal procedure, as shown in FIG. 9, is performed. Once the physician gains access, the stomach muscle is injected with botulinum toxin at administration sites 50 (referring to FIGS. 4A, 4B and 9). FIG. 4A shows targeted administration sites 50 in the area where incisions and sutures are made, while FIG. 4B shows more generalized administration sites 50.

The gastric bypass procedure is performed after the surgeon determines that the stomach is adequately relaxed by the administration of a botulinum toxin. The gastric bypass procedure takes less time as compared to the same procedure where the stomach is not relaxed by the administration of a botulinum toxin, as the surgeon can manipulate the stomach more easily. This allows the physician to perform the procedure more quickly. Moreover, the recovery time from the gastric bypass procedure performed after the stomach is relaxed by the administration of a botulinum toxin is faster as compared to that of the same procedure where the stomach is not relaxed by the administration of a botulinum toxin.

### Example 10

In this tenth example, a method for making a botulinum toxin tablet for ingestion is discussed.

A botulinum toxin can be compounded as an oral formulation for release of the toxin active ingredient into the stomach or duodenum. This is easily accomplished by mixing with a mortar and pestle (at room temperature without addition of any water or saline) 50 units of a commercially available lyophilized botulinum toxin powder, such as non-reconstituted BOTOX® (or 200 units of DYSPORT® powder) with a biodegradable carrier such as flour or sugar. Alternately, the botulinum toxin can be mixed by homogenization or sonication to form a fine dispersion of the powdered toxin in the carrier. The mixture can then compressed with a tablet making machine (such as the tablet press available from Scheu & Kniss, 1500 W. Ormsby Ave, Louisville, Ky. 40210) to make an ingestible tablet. Alternately, the toxin can be formulated with gelatin by well known methodologies to make an ingestible geltab.

All references, articles, publications and patents and patent applications cited herein are incorporated by reference in their entireties.

Although the present invention has been described in detail with regard to certain preferred methods, other embodiments, versions, and modifications within the scope of the present invention are possible. For example, a wide variety of Clostridial toxins can be effectively used in the methods of the present invention. Additionally, the present invention includes oral formulations where two or more botulinum toxins are administered concurrently or consecutively via the oral formulation. For example, botulinum toxin type A can be administered via an oral formulation until a loss of clinical response or neutralizing antibodies develop, followed by administration also by suitable oral formulation of a botulinum toxin type B or E. Alternately, a combination of any two or more of the botulinum serotypes A-G can be locally administered to control the onset and duration of the desired therapeutic result. Furthermore, non-Clostridial toxin compounds can be administered prior to, concurrently with or subsequent to administration of the Clostridial toxin via oral formulation so as to provide an adjunct effect such as enhanced or a more rapid onset of denervation before the Clostridial toxin, such as a botulinum toxin, begins to exert its therapeutic effect.

What is claimed is:

1. A method for reducing side effects associated with insertion and deployment of an intragastric balloon in a patient's stomach, the method comprising the steps of:
(a) administering a neurotoxin to a stomach tissue of the patient, and
(b) deploying the intragastric balloon in the stomach of the patient.

2. The method of claim 1, wherein the neurotoxin is administering locally.

3. The method of claim 2, wherein the neurotoxin is administered locally at a site or in a vicinity of the site where the intragastric balloon contacts the stomach.

4. The method of claim 2, wherein the neurotoxin is administered locally to an upper part of the stomach.

5. The method of claim 1, wherein the neurotoxin is administered orally.

6. The method of claim 1, wherein the step of administering the neurotoxin is carried out prior to the step of deploying the intragastric balloon in the stomach.

7. The method of claim 1 further comprising the step of adjusting the volume of the intragastric balloon in situ.

8. The method of claim 7, wherein the step of administering the neurotoxin is carried out prior to the step of adjusting the volume of the intragastric balloon in situ.

9. The method of claim 1, wherein the neurotoxin is a botulinum toxin selected from the group consisting of botulinum toxins types A, B, C₁, D, E, F and G.

10. The method of claim 1, wherein the neurotoxin is a botulinum toxin type A.

11. The method of claim 1, wherein the stomach tissue is a stomach smooth muscle.
